(19) United States
(12) Patent Application Publication
(10) Pub. No.: US 2025/0101504 A1
(43) Pub. Date: Mar. 27, 2025

(54) METHODS OF MEASURING MISLOCALIZATION OF AN ANALYTE

(71) Applicant: 10x Genomics, Inc., Pleasanton, CA (US)

(72) Inventors: Monica Nagendran, Pleasanton, CA (US); Jonathan Cheng, Pleasanton, CA (US); Eswar Prasad Ramachandran Iyer, Oakland, CA (US)

(21) Appl. No.: 18/976,515

(22) Filed: Dec. 11, 2024

Related U.S. Application Data
(63) Continuation of application No. 18/087,440, filed on Dec. 22, 2022, now Pat. No. 12,203,134.
Application No. 18/087,440 is a continuation of application No. PCT/US2022/024646, filed on Apr. 13, 2022.
(60) Provisional application No. 63/174,789, filed on Apr. 14, 2021.

(51) Int. Cl. C12Q 1/6841; C12Q 1/6806; C12Q 1/6844

(52) U.S. Cl. CPC C12Q 1/6841; C12Q 1/6806; C12Q 1/6844; C12Q 2600/16

(57) ABSTRACT

Provided herein are methods for determining the mislocalization of an analyte by capturing the analyte on an array and measuring the mislocalization distance.

### CROSS-REFERENCE TO RELATED APPLICATIONS

This application is a continuation of U.S. patent application Ser. No. 18/087,440, filed on a continuation of International Application Dec. 22, 2022, which is PCT/US2022/024646, with an international filing date of Apr. 13, 2022, which claims the benefit of U.S. Provisional Patent Application No. 63/174,789, filed Apr. 14, 2021. The contents of these priority applications are incorporated herein by reference in their entireties.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

Generally, spatial analysis requires determining the sequence of the analyte sequence or a complement thereof and the sequence of the spatial barcode or a complement thereof in order to identify the spatial location of the analyte in the original biological sample. Typically, this requires sequencing which can be time and resource intensive. Therefore, there is a need to assess analyte (biological sample) spatial preservation and presence prior to traditional spatial analysis.

### SUMMARY

In one aspect, this disclosure features methods of determining analyte mislocalization within a biological sample, the method including: (a) obtaining a first image of the biological sample; (b) hybridizing the analyte to a capture probe on an array, wherein the array includes a plurality of capture probes, wherein a capture probe of the plurality of capture probe includes a capture domain; (c) extending the capture probe using the analyte as a template, thereby generating an extended capture probe; (d) hybridizing a padlock probe or a snail probe to the extended capture probe; (e) circularizing the padlock probe or the snail probe; (f) amplifying the padlock probe or the snail probe, thereby generating an amplified circularized padlock probe or an amplified circularized snail probe; (g) hybridizing a plurality of detection probes to the amplified circularized padlock probe or the amplified circularized snail probe, wherein a detection probe from the plurality of detection probes comprises: a sequence that is substantially complementary to a sequence of the padlock probe or the snail probe, or a complement thereof, and a detectable label; (h) obtaining a second image of the biological sample and detecting a signal from the plurality of detection probes in the second image; and (i) determining mislocalization of the analyte within the biological sample by comparing the first image of the biological sample to the second image of the biological sample.

Also provided herein is a method of determining mislocalization of an analyte within a biological sample, the method comprising: (a) obtaining a first image of the biological sample; (b) hybridizing an analyte derived molecule to a capture domain of a capture probe on an array, wherein the array comprises a plurality of capture probes, wherein the plurality of capture probes comprise a capture domain; (c) hybridizing a padlock probe or a snail probe to the analyte derived molecule; (d) circularizing the padlock probe or the snail probe; (e) amplifying the padlock probe or the snail probe, thereby generating an amplified circularized padlock probe or an amplified circularized snail probe; (f) hybridizing a plurality of detection probes to the amplified circularized padlock probe or the amplified circularized snail probe, wherein a detection probe from the plurality of detection probes comprises: a sequence that is substantially complementary to a sequence of the padlock probe or the snail probe, or a complement thereof, and a detectable label; (g) obtaining a second image of the biological sample and detecting a signal from the plurality of detection probes in the second image; and (h) determining mislocalization of the analyte within the biological sample by comparing the first image of the biological sample to the second image of the biological sample.

In some instances, the analyte derived molecule is a ligation product. In some instances, the method further includes, prior to step (b): contacting a plurality of first probes and second probes to the biological sample on the array, wherein the plurality of first probes and second probes target a plurality of nucleic acids in the biological sample, wherein a first probe and a second probe of the plurality comprise sequences that are substantially complementary to the analyte, wherein the analyte is a target nucleic acid, and wherein the second probe comprises a capture probe capture domain sequence that is complementary to all or a portion of the capture domain; hybridizing the first probe and the second probe to the target nucleic acid; generating a ligation product by ligating the first probe and the second probe; and releasing the ligation product from the target nucleic acid.

In some instances, the first probe and the second probe are substantially complementary to adjacent sequences of the target nucleic acid. In some instances, the first probe and the second probe hybridize to sequences that are not adjacent to each other on the target nucleic acid, and wherein the first probe is extended with a DNA polymerase, thereby (i) filling in a gap between the first probe and the second probe and (ii) generating an extended first probe. In some instances, the first probe further comprises a primer sequence. In some instances, the first probe and/or the second probe is a DNA probe. In some instances, the releasing the ligation product from the target nucleic acid comprises contacting the biological sample with an endoribonuclease, optionally wherein the endoribonuclease is an RNase H enzyme.

In some instances, the analyte derived molecule is an analyte capture agent. In some instances, the methods further include, prior to step (b): contacting the biological sample with a plurality of analyte capture agents, wherein the analyte capture agent comprises an analyte binding moiety and an oligonucleotide comprising an analyte binding moiety barcode and an analyte capture sequence, wherein the analyte capture sequence comprises a sequence complementary to the capture domain; and binding the analyte binding moiety of the analyte capture agent to the analyte, wherein the analyte is a protein. In some instances, step (b) comprises hybridizing the analyte capture sequence to the capture domain.

In some instances, the padlock probe or the snail probe comprises: (i) a first sequence that is substantially complementary to a first portion of the extended capture probe, the analyte derived molecule, or a complement thereof; (ii) a backbone sequence; and (iii) a second sequence that is substantially complementary to a second portion of the extended capture probe, the analyte derived molecule, or a complement thereof.

In some embodiments, the first sequence and the second sequence are substantially complementary to adjacent sequences of the extended capture probe, the analyte derived molecule, or a complement thereof. In some embodiments, the first sequence and the second sequence are substantially complementary to sequences of the extended capture probe, the analyte derived molecule, or a complement thereof, that are about 2 to 50 nucleotides apart.

In some embodiments, circularizing the padlock probe or the snail probe includes filling in a gap between the first sequence that is substantially complementary to a first portion of the analyte and the second sequence that is substantially complementary to a second portion of the analyte using a polymerase.

In some embodiments, the backbone sequence includes a backbone barcode sequence.

In some embodiments, circularizing the padlock probe or the snail probe includes ligating the padlock probe or the snail probe.

In some embodiments, the ligating includes enzymatic ligation or chemical ligation (e.g., click chemistry). In some embodiments, the enzymatic ligation utilizes ligase. In some embodiments, ligase includes a T4 DNA ligase.

In some embodiments, the amplifying includes rolling circle amplification (RCA). In some embodiments, the amplifying includes hybridizing one or more amplification primers to the padlock probe or the snail probe; and amplifying the padlock probe or the snail probe with a padlock probe or snail probe polymerase. In some embodiments, the padlock probe or snail probe polymerase has strand displacement activity. In some embodiments, the padlock probe or snail probe polymerase is a Phi29 DNA polymerase.

In some embodiments, the one or more amplification primers are substantially complementary to a portion of the padlock probe or the snail probe.

In some embodiments, the detection probe is about 10 to about 60 nucleotides in length.

In some embodiments, the detectable label includes a fluorophore.

In some embodiments, the detection probe is substantially complementary to a portion of the first sequence that is substantially complementary to a first portion of the analyte.

In some embodiments, the detection probe is substantially complementary to a portion of the second sequence that is substantially complementary to a second portion of the analyte.

In some embodiments, the detection probe is substantially complementary to a portion of the backbone sequence.

In some embodiments, the detection probe is substantially complementary to a portion of the analyte or a complement thereof.

In some embodiments, the extending step includes extending the capture probe using the analyte as the template. In some embodiments, the extending includes using a reverse transcriptase. In some embodiments, the reverse transcriptase includes a Moloney murine leukemia virus reverse transcriptase. In some embodiments, extending the capture probe includes using fluorescently tagged nucleotides to reverse transcribe the extended capture probe.

In some embodiments, the biological sample is permeabilized using a permeabilization agent. In some embodiments, the permeabilization agent includes proteinase K. In some embodiments, the permeabilization agent includes pepsin.

In some embodiments, the biological sample is permeabilized at room temperature.

In some embodiments, the biological sample is permeabilized at 37° C.

In some embodiments, the biological sample is permeabilized for about 1 minute to about 30 minutes.

In some embodiments, the method also includes varying permeabilization time to determine an optimal permeabilization time for minimizing analyte mislocalization in the biological sample.

In some embodiments, the method also includes varying permeabilization temperature to determine an optimal permeabilization temperature for minimizing analyte mislocalization in the biological sample.

In some embodiments, determining the mislocalization of the analyte includes measuring the signal intensity of the plurality of detection probes and comparing the signal intensity to a first image of the biological sample. In some embodiments, signal intensity in the image of the biological sample includes a single cell or cell type that expresses a biomarker that is detected when the biological sample is stained.

In some embodiments, the biological sample is stained prior to obtaining an image of the biological sample. In some embodiments, the biological sample is stained using immunofluorescence or immunohistochemistry techniques. In some embodiments, the biological sample is stained using hematoxylin and eosin (H&E).

In some embodiments, the image is a fluorescent image or a brightfield image.

In some embodiments, the image is obtained using a fluorescent microscope, a confocal microscope, or a brightfield microscope.

In some embodiments, the capture probe is affixed to the substrate at a 5′ end of the capture probe.

In some embodiments, the plurality of capture probes are uniformly distributed on a surface of the substrate.

In some embodiments, the capture domain of the capture probe includes a homopolymeric sequence. In some embodiments, the capture domain of the capture probe includes a poly(T) sequence. In some embodiments, the capture domain of the capture probe includes a non-homopolymeric sequence. In some embodiments, the non-homopolymeric sequence includes a sequence substantially complementary to the analyte.

In some embodiments, the biological sample includes a FFPE sample. In some embodiments, the biological sample includes a fresh frozen sample. In some embodiments, the biological sample includes fixed or live cells. In some embodiments, the biological sample is from a mammal. In some embodiments, the biological sample is from a human, a mouse, or a rat.

In some embodiments, the biological sample includes a tissue section. In some embodiments, the tissue section is about 2.5 μm to about 20 μm in thickness.

In some embodiments, the method also includes varying the thickness of the tissue sample to determine an optimal tissue thickness to minimize mislocalization in the biological sample.

In some embodiments, the analyte is RNA. In some embodiments, the RNA is mRNA. In some embodiments, the analyte is a protein. In some embodiments, the protein is a cell surface marker. In some embodiments, the protein is an immune cell receptor.

In some embodiments, the method also includes obtaining a second biological sample. In some embodiments, the second biological sample is a second tissue section of the biological sample.

In some embodiments, the method also includes determining abundance and/or location of an analyte in the second biological sample, wherein the determining includes: (a) contacting a spatial array with the second biological sample, wherein the spatial array includes a plurality of spatial capture probes, wherein a spatial capture probe of the plurality of spatial capture probes includes a capture domain and a spatial barcode; (b) hybridizing the analyte to the spatial capture probe; and (c) determining (i) all or a part of the sequence of the analyte, or a complement thereof, and (ii) the sequence of the spatial barcode, or a complement thereof, and using the determined sequence of (i) and (ii) to determine the abundance and/or the location of the analyte in the biological sample. In some embodiments, the spatial capture probe further includes one or more functional domains, a unique molecular identifier (UMI), a cleavage domain, or combinations thereof. In some embodiments, the method also includes extending the spatial capture probe via a nucleic acid extension reaction using the analyte as a template to generate an extended spatial capture probe including the spatial capture probe and the reverse complement of the analyte. In some embodiments, the determining step (c) includes amplifying all or part of the analyte specifically bound to the spatial capture domain, or a complement thereof. In some embodiments, the determining step (c) includes sequencing. In some embodiments, the method can include imaging the biological sample.

In another aspect, this disclosure features kits, where the kits include: (a) an array including a plurality of capture probes, wherein a capture probe of the plurality of capture probes includes a capture domain; (b) a plurality of padlock probes and/or snail probes, wherein a padlock probe or a snail probe of the plurality of padlock probes and/or snail probes includes: (i) a first sequence that is substantially complementary to a first portion of an analyte, or a complement thereof, (ii) a backbone sequence, and (iii) a second sequence that is substantially complementary to a second portion of the analyte, or a complement thereof; (c) one or more enzymes selected from a ligase, a polymerase, a reverse transcriptase, or combinations thereof; (d) a plurality of detection probes; and (e) instructions for performing any one of the methods of described herein.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a capture probe, as described herein.

FIG. 2 is a schematic illustrating a cleavable capture probe, wherein the cleaved capture probe can enter into a non-permeabilized cell and bind to target analytes within the cell.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce a spatially-barcoded cells or cellular contents.

FIG. 7A is a schematic diagram of an exemplary substrate 702 and biological sample 701.

FIG. 7B is a schematic illustrating a cross-sectional view of the exemplary substrate having capture probes 703 and biological sample 701 having analytes 705 in FIG. 7A.

FIG. 8A is a schematic diagram of an exemplary padlock oligonucleotide 800.

FIG. 8B is a schematic diagram of an exemplary padlock oligonucleotide 815 hybridized to an extended capture probe 805 bound to a substrate.

FIG. 8C is a schematic diagram of an exemplary primer 807 hybridized to an exemplary circularized padlock oligonucleotide 815.

FIG. 8D is a schematic illustrating the product 810 of a rolling circle amplification reaction (i.e., amplified circularized padlock oligonucleotide).

FIG. 8E is a schematic showing a plurality of detection probes 811 bound to multiple regions of the amplified circularized padlock oligonucleotide 810.

FIG. 9 is a schematic illustrating detected signal from a plurality of amplified circularized padlock oligonucleotides shown as regions 1 (circles), 2 (stars), 3 (rectangles), and 4 (diamonds).

FIGS. 10A-10D show gene expression heat maps for Scgb1a1 across serial mouse lung tissue sections following different permeabilization conditions.

FIG. 11 shows fluorescent images of mouse lung: upper left-DAPI, lower left-auto fluorescence, upper right-cDNA footprint from spatial array analysis, and lower right-fluorescent probes hybridized to the product of an RCA reaction. Arrows indicate three exemplary lung tissue airways.

FIGS. 12A-12E shows immunofluorescence images of mouse lung. FIGS. 12A-12C show magnified views of images from FIG. 11. Arrows indicate the same three exemplary lung tissue airways as indicated in FIG. 11. FIGS. 12D-12E show single molecule FISH (smFISH) images for Scgb1a1 transcripts (FIG. 12E) and Scgb1a1 transcripts merged with DAPI staining (FIG. 12D).

FIGS. 13A-13B show representative images of (FIG. 13A) DAPI (i.e., nuclear) staining and Prox1 mRNA expression after RCA reaction and (FIG. 13B) Prox1 mRNA expression after RCA reaction alone in fresh frozen mouse brain samples.

FIG. 14 shows an exemplary templated ligation and RCA workflow.

FIG. 15 shows a schematic of the methods of templated ligation and RCA using a snail probe.

### DETAILED DESCRIPTION

### I. Introduction

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, an analyte derived molecule such as a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Some array-based spatial analysis methods involve the transfer of one or more analytes from a biological sample to an array of features on a substrate, where typically each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for amplification, or a flow cell attachment sequence, such as for next-generation sequencing (NGS)).

In some instance, the capture domain is designed to detect one or more specific analytes of interest. For example, a capture domain can be designed so that it comprises a sequence that is complementary or substantially complementary to one analyte of interest.

Thus, the presence of a single analyte can be detected. Alternatively, the capture domain can be designed so that it comprises a sequence that is complementary or substantially complementary to a conserved region of multiple related analytes. In some instances, the multiple related analytes are analytes that function in the same or similar cellular pathways or that have conserved homology and/or function. The design of the capture probe can be determined based on the intent of the user and can be any sequence that can be used to detect an analyte of interest. In some embodiments, the capture domain sequence can therefore be random, degenerate, defined (e.g., gene or protein specific) or combinations thereof, depending on the target analyte(s) of interest.

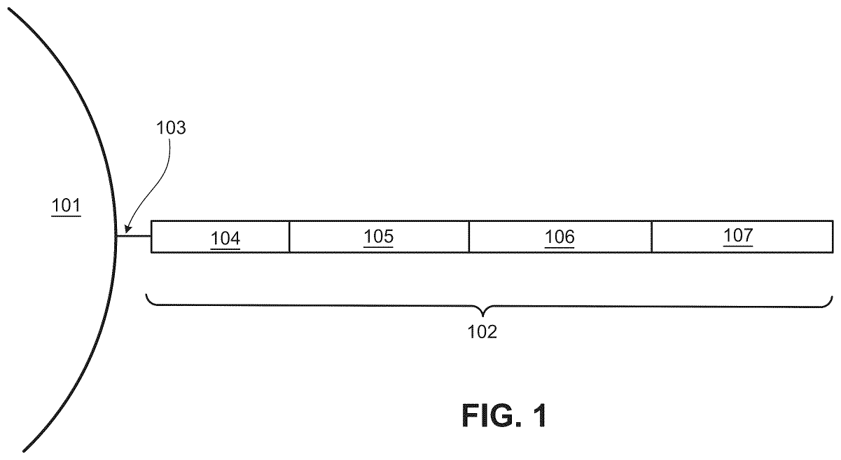

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and

Oxford Nanopore sequencing. Further, in some embodiments, functional sequences can be selected for compatibility with other sequencing systems, including non-commercialized sequencing systems.

In some embodiments, the spatial barcode 105 and functional sequence 104 is common to all of the probes attached to a given feature. In some embodiments, the UMI sequence 106 of a capture probe attached to a given feature is different from the UMI sequence of a different capture probe attached to the given feature.

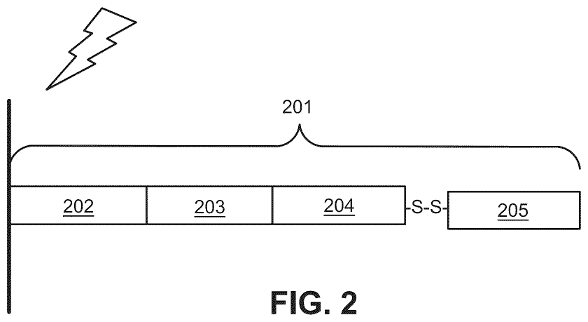

FIG. 2 is a schematic illustrating a cleavable capture probe, wherein the cleaved capture probe can enter into a non-permeabilized cell and bind to analytes within the cell. The capture probe 201 contains a cleavage domain 202, a cell penetrating peptide 203, a reporter molecule 204, and a disulfide bond (—S—S—). 205 represents all other parts of a capture probe, for example a spatial barcode and/or a capture domain.

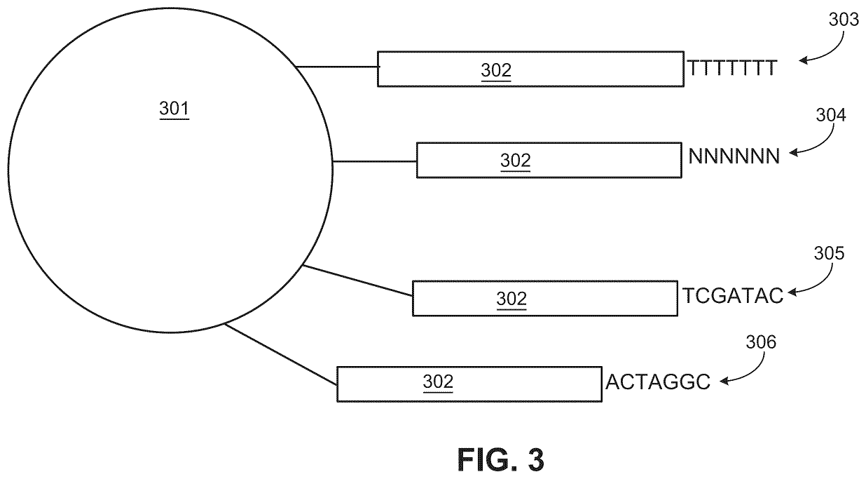

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode 302, but have different capture domains designed to associate the spatial barcode of the feature with more than one target analyte. For example, a feature may be coupled to four different types of spatially-barcoded capture probes, each type of spatially-barcoded capture probe possessing the same spatial barcode 302. One type of capture probe associated with the feature includes the spatial barcode 302 in combination with a poly(T) capture domain 303, designed to capture mRNA target analytes. A second type of capture probe associated with the feature includes the same spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

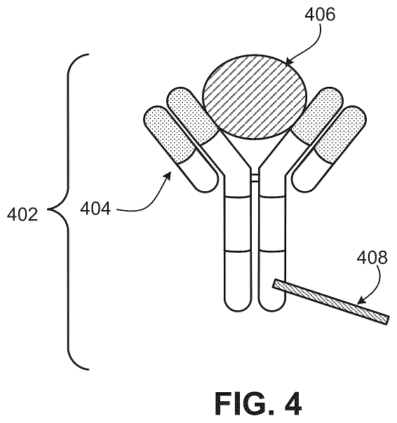

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

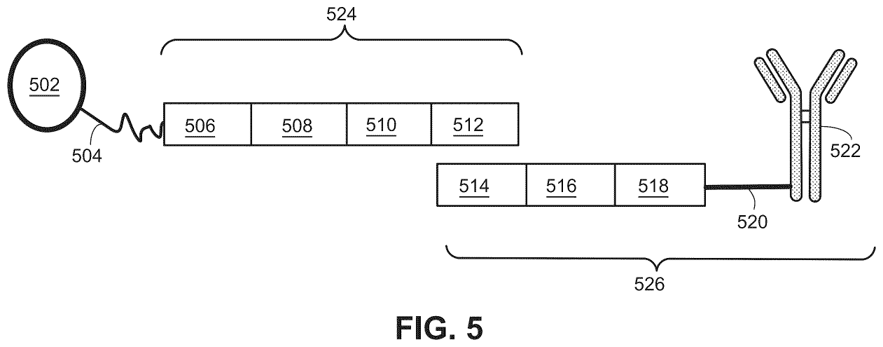

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

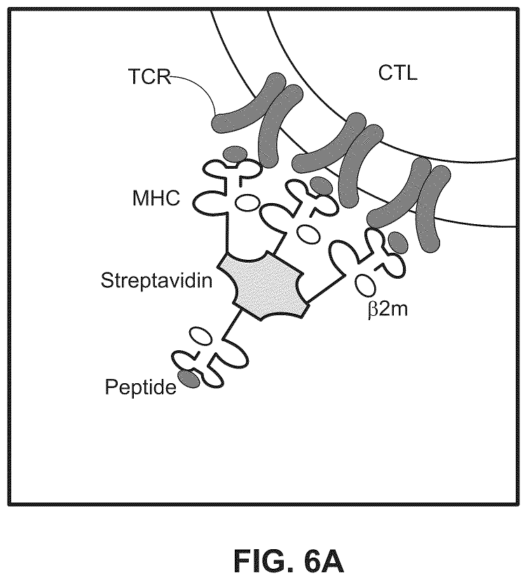

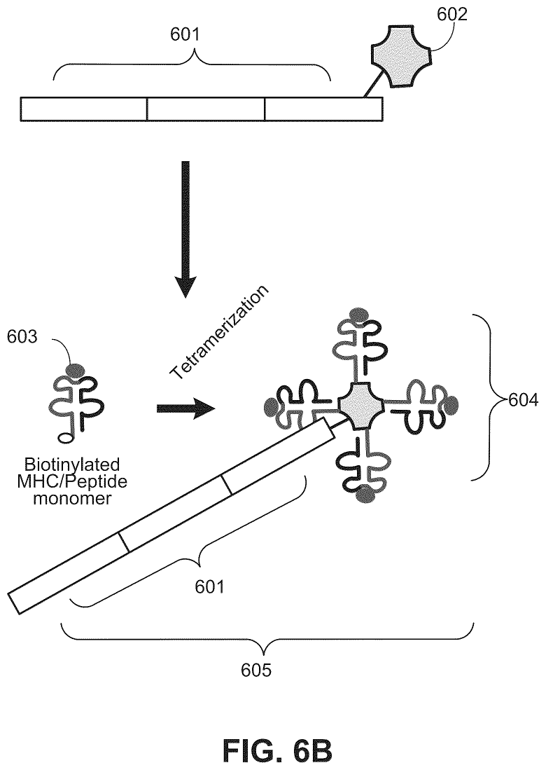

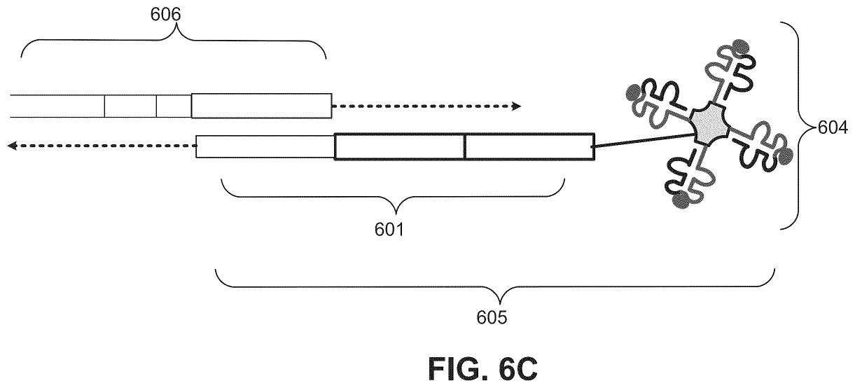

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a T-Cell Receptor (TCR) such that the streptavidin binds to a target T-cell via multiple MCH/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC molecules 604 are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions (see dashed lines adjacent to 606 and 601) such that constructs comprising sequences that correspond to each of the two spatial barcode sequences (the spatial barcode associated with the capture probe, and the barcode associated with the MHC-oligonucleotide complex) are generated. In some cases, one or both of these corresponding sequences may be a complement of the original sequence in capture probe 606 or capture agent barcode domain 601. In other embodiments, the capture probe and the capture agent barcode domain are ligated together. The resulting construct can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using pMHC libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample. In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for template extension.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe, a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject;

identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities including capture probes, used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the tissue. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the spatial array. Where necessary, the spatial arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

The sandwich process is described in PCT Patent Application Publication No. WO 2020/123320, which is incorporated by reference in its entirety.

### II. Methods and Compositions for Measuring Analyte Mislocalization Using Rolling Circle Amplification

### (a) Introduction

Disclosed herein are methods for determining analyte mislocalization in a biological sample. As used herein, analyte mislocalization is determined by measuring the diffusion, or the migration of an analyte (e.g., an mRNA molecule), from a biological sample to an array comprising capture probes. In instances in which an analyte migrates to the closest probe in proximity to the analyte (i.e., migrating in a line that is perpendicular to the surface of the array, there is minimal to zero mislocalization. On the other hand, an analyte may migrate to probes that are not the probe closest in proximity to the analyte's original position in the biological sample, resulting in increased analyte mislocalization. In this instance, the distance between the location of the closest probe and the probe that ultimately captures the analyte is the mislocalization distance. Ideally, the mislocalization distance when transferring an analyte from a biological sample to the capture probe is zero or near-zero. However, in several instances, the mislocalization distance is not zero. It is appreciated by this disclosure that limiting the mislocalization distance for each sample tested (e.g., by varying parameters of permeabilization and by capturing only one or a few analytes) would increase resolution for determining the location of an analyte in a biological sample. Described herein are methods, kits, and compositions to determine the mislocalization distance of analytes using a fluorescent readout via rolling circle amplification of the captured analyte. The method allows a user to directly determine mislocalization of an analyte in a biological sample. Once the mislocalization distance parameters are optimized, a user can utilize these parameters on a related sample (e.g., in a serial section) and look more globally (e.g., at the entire transcriptome or proteome) at analyte expression. It is to be appreciated that different tissue types (e.g., porous, non-porous and dense tissue samples) may each have distinct analyte mislocalization profiles.

Thus, disclosed herein are methods of determining analyte mislocalization within a biological sample, the method including: (a) obtaining a first image of the biological sample; (b) hybridizing the analyte or analyte derived molecule to a capture probe on an array, wherein the array includes a plurality of capture probes, wherein a capture probe of the plurality of capture probe includes a capture domain; (c) optionally extending the capture probe using the analyte as a template, thereby generating an extended capture probe; (d) hybridizing a padlock probe or a snail probe to the analyte derived molecule or extended capture probe; (e) circularizing the padlock probe or the snail probe; (f) amplifying the padlock probe or the snail probe, thereby generating an amplified circularized padlock probe or an amplified circularized snail probe; (g) hybridizing a plurality of detection probes to the amplified circularized padlock probe or the amplified circularized snail probe, wherein a detection probe from the plurality of detection probes comprises: a sequence that is substantially complementary to a sequence of the padlock probe or the snail probe, or a complement thereof, and a detectable label; (h) obtaining a second image of the biological sample and detecting a signal from the plurality of detection probes in the second image; and (i) determining mislocalization of the analyte within the biological sample by comparing the first image of the biological sample to the second image of the biological sample.

Also disclosed herein are compositions and kits that are used in the methods.

### (b) Methods of Measuring Analyte Mislocalization by Direct Capture of Analytes

In some embodiments, disclosed herein are methods of determining mislocalization of an analyte within a biological sample by capture of an analyte directly onto a substrate (e.g., hybridization of an analyte to a capture probe). In some instances, the analyte is RNA, such as mRNA. After obtaining a first image of the biological sample, the analyte hybridizes to a capture domain of a capture probe on an array, which comprises a plurality of capture probes, wherein the plurality of capture probes comprise a capture domain.

After hybridization, the capture probe is extended using a polymerase (e.g., a reverse transcriptase) to generate an extended capture probe. In some instances, extending the capture probe comprises using fluorescently tagged nucleotides to reverse transcribe the analyte. Either oligonucleotides containing fluorescently tagged nucleotides or individual fluorescently tagged nucleotides can be used. For instance, in some embodiments, individual fluorescently tagged nucleotides are used. The fluorescently tagged nucleotides can include a mixture of dNTPs comprises dATP, dTTP, dCTP, and dGTP, wherein at least one of the dATP, dTTP, dCTP, and dGTP is labeled with a fluorophore. In some instances, the capture probe is extended using non-labelled dNTP's to generate an extended capture probe lacking fluorescently tagged nucleotides.

At this point, the extended capture probe includes the capture probe and a complement of the analyte (or a portion thereof). Once the extended capture probe (e.g., cDNA molecule) is generated, the analyte (e.g., mRNA) can be dissociated from the capture probe. In some instances, this is performed using a solution comprising KOH. After dissociation of the analyte, the extended capture probe—which is single stranded—can interact with a padlock probe or a snail probe via hybridization. After hybridization of the padlock probe or snail probe, the padlock probe or snail probe is circularized, amplified, and detected using labeled detection probes, as described herein. A second image is obtained to determine the signal intensity from the plurality of detection probes in the second image. Comparing the first image and the second image allows one to determine mislocalization of the analyte within the biological sample by comparing the first image of the biological sample to the second image of the biological sample. For example, analyte mislocalization can be determined by comparing the expression of a cellular marker (e.g., for a particular cell type or morphology) found in the first image with the same, or similar, cellular marker in the second image. For example, Prox1 is expressed in several brain regions of mammals including the cortex, dentate gyrus (DG) region of the hippocampus, cerebellum, and hypothalamus. Since Prox1 expression is known to occur in the DG region of the hippocampus, mRNA transcripts encoding the Prox1 protein will also be present in the DG region of the hippocampus. Comparing and/or aligning the expression profile of the cellular markers allows one to determine (e.g., calculate) the relative distance an analyte has migrated from its original position in the biological sample.

### (c) Methods of Measuring Analyte Mislocalization by Capture of Analyte Proxies

### (i) Templated Ligation

In some embodiments, disclosed herein are methods that utilizes a nucleic acid proxy to determine mislocalization of an analyte. In some instances, the proxy is a ligation product derived from templated ligation. Methods of RNA-templated ligation (RTL) are disclosed in US 2021/0348221, US 2021/0285046, and WO 2021/133849, each of which is incorporated by reference in its entirety.

In an exemplary embodiment of the disclosure, provided are methods to determine mislocalization of an analyte on the array using templated ligation. In some instances, the methods include contacting a biological sample with array of capture probes. In some instances, the array is on a substrate and the array includes a plurality of capture probes, wherein a capture probe of the plurality includes a capture domain. After placing the biological sample on the array, the biological sample is contacted with a first probe and a second probe, wherein the first probe and the second probe each include one or more sequences that are substantially complementary to sequences of the analyte, and wherein the second probe includes a capture probe capture domain; the first probe and the second probe hybridize to complementary sequences in the analyte. After hybridization, a ligation product comprising the first probe and the second probe is generated, and the ligation product is released from the analyte. The liberated ligation product is then available to hybridize to the capture domain of a probe on the array. After capture on the array, padlock probes or snail probes described in section (II)(d) below can be used to determine mislocalization of the analyte on the array.

In another non-limiting example, a biological sample is deparaffinized, stained, and imaged. After destaining and decrosslinking, probes are added to the sample and hybridized to an analyte. In some instances, the probes are DNA probes. In some instances, the probes are diribo-containing probes. Probes are ligated to form a ligation product and released from the biological sample, for example using an endonuclease such as RNAse H. The ligation product can be captured on an array by a capture probe, where the ligation product can hybridize to a padlock probe or snail probe as described in section (II)(d) below.

In some embodiments, the ligation product includes a capture probe capture domain, which can hybridize to a capture probe (e.g., a capture probe immobilized, directly or indirectly, on a substrate). In some embodiments, methods provided herein include contacting a biological sample with a substrate, wherein the capture probe is affixed to the substrate (e.g., immobilized to the substrate, directly or indirectly). In some embodiments, the capture probe capture domain of the ligated product specifically binds to the capture domain. The capture probe can also include a unique molecular identifier (UMI), a spatial barcode, a functional sequence, a cleavage domain, or a combination thereof.

The methods provided herein utilize probe pairs (or probe sets; the terms are interchangeable). In some instances, the probe pairs are designed so that each probe hybridizes to a sequence in an analyte that is specific to the analyte (e.g., compared to the entire transcriptome). That is, in some instances, a single probe pair can be specific to a single analyte.

In other embodiments, probes can be designed so that one of the probes of a pair is a probe that hybridizes to a specific sequence. Then, the other probe can be designed to detect a mutation of interest. Accordingly, in some instances, multiple second probes can be designed and can vary so that each probe binds to a specific sequence. For example, a second probe can be designed to hybridize to a wild-type sequence, and another second probe can be designed to hybridize (and therefore detect) a mutated sequence. Thus, in some instances, a probe set can include one first probe and two second probes (or vice versa).

On the other hand, in some instances, probes can be designed so that they cover conserved regions of an analyte. Thus, in some instances, a probe (or probe pair can hybridize to similar analytes in a biological sample (e.g., to detect conserved or similar analytes) or in different biological samples (e.g., across different species).

In some embodiments, probe sets cover all or nearly all of a genome (e.g., human genome). In instances where probe sets are designed to cover an entire genome (e.g., the human genome), the methods disclosed herein can detect analytes in an unbiased manner. In some instances, one probe oligonucleotide pair is designed to cover one analyte (e.g., transcript). In some instances, more than one probe oligonucleotide pair (e.g., a probe pair comprising a first probe and a second probe) is designed to cover one analyte (e.g., transcript). For example, at least two, three, four, five, six, seven, eight, nine, ten, or more probe sets can be used to hybridize to a single analyte. Factors to consider when designing probes is presence of variants (e.g., SNPs, mutations) or multiple isoforms expressed by a single gene. In some instances, the probe oligonucleotide pair does not hybridize to the entire analyte (e.g., a transcript), but instead the probe oligonucleotide pair hybridizes to a portion of the entire analyte (e.g., transcript).

In some instances, about 100, 200, 300, 400, 500, 600, 700, 800, 900, 1000, 2000, 3000, 4000, 5000, 10000, 15000, 20000, or more probe oligonucleotides pair (e.g., a probe pair comprising a first probe and a second probe) are used in the methods described herein. In some instances, about 20,000 probe oligonucleotides pair are used in the methods described herein. In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, or about 1000 analytes.

In some embodiments, a first probe and/or the second probe includes a nucleic acid sequence that is about 10 nucleotides to about 100 nucleotides (e.g., a sequence of about 10 nucleotides to about 90 nucleotides, about 10 nucleotides to about 80 nucleotides, about 10 nucleotides to about 70 nucleotides, about 10 nucleotides to about 60 nucleotides, about 10 nucleotides to about 50 nucleotides, about 10 nucleotides to about 40 nucleotides, about 10 nucleotides to about 30 nucleotides, about 10 nucleotides to about 20 nucleotides, about 20 nucleotides to about 100 nucleotides, about 20 nucleotides to about 90 nucleotides, about 20 nucleotides to about 80 nucleotides, about 20 nucleotides to about 70 nucleotides, about 20 nucleotides to about 60 nucleotides, about 20 nucleotides to about 50 nucleotides, about 20 nucleotides to about 40 nucleotides, about 20 nucleotides to about 30 nucleotides, about 30 nucleotides to about 100 nucleotides, about 30 nucleotides to about 90 nucleotides, about 30 nucleotides to about 80 nucleotides, about 30 nucleotides to about 70 nucleotides, about 30 nucleotides to about 60 nucleotides, about 30 nucleotides to about 50 nucleotides, about 30 nucleotides to about 40 nucleotides, about 40 nucleotides to about 100 nucleotides, about 40 nucleotides to about 90 nucleotides, about 40 nucleotides to about 80 nucleotides, about 40 nucleotides to about 70 nucleotides, about 40 nucleotides to about 60 nucleotides, about 40 nucleotides to about 50 nucleotides, about 50 nucleotides to about 100 nucleotides, about 50 nucleotides to about 90 nucleotides, about 50 nucleotides to about 80 nucleotides, about 50 nucleotides to about 70 nucleotides, about 50 nucleotides to about 60 nucleotides, about 60 nucleotides to about 100 nucleotides, about 60 nucleotides to about 90 nucleotides, about 60 nucleotides to about 80 nucleotides, about 60 nucleotides to about 70 nucleotides, about 70 nucleotides to about 100 nucleotides, about 70 nucleotides to about 90 nucleotides, about 70 nucleotides to about 80 nucleotides, about 80 nucleotides to about 100 nucleotides, about 80 nucleotides to about 90 nucleotides, or about 90 nucleotides to about 100 nucleotides).

### (ii) Protein Detection

In some embodiments, proteins in a biological sample can be detected using analyte capture agents, as described herein. In some embodiments, the analyte capture agents are contacted with the biological sample before the biological sample is contacted with an array. In some embodiments, the analyte capture agents are contacted with the biological sample after the biological sample is contacted with the array. In some embodiments, an analyte binding moiety of the analyte capture agent interacts (e.g., binds) with an analyte (e.g., protein) in a biological sample. In some embodiments, the analyte binding moiety is an antibody, aptamer or antigen-binding fragment.

Analyte capture agents can also include a conjugated oligonucleotide that can comprise one or more domains. For example, the conjugated oligonucleotide can include an analyte binding moiety barcode and an analyte capture sequence. In some embodiments, the analyte binding moiety barcode, or a complement thereof, refers to (e.g., identifies) a barcode that is associated with or otherwise identifies the analyte binding moiety. In some embodiments, the conjugated oligonucleotide can include an analyte capture sequence. In some embodiments, the analyte capture sequence is capable of interacting with (e.g., hybridizing) a capture domain of a capture probe on a substrate.

In some instances, an extended capture probe is generated using the analyte capture sequence as a template. In this instance, the padlock or snail probes (described in the Section (II)(d) below) can hybridize to the extended capture probe.

In some instances, no extended capture probe is generated using the analyte capture sequence as a template. In this instance, the padlock or snail probes (described in the Section (II)(d) below) hybridize directly to the analyte capture sequence.

In some embodiments, analyte capture agents are capable of binding to analytes present inside a cell. In some embodiments, analyte capture agents are capable of binding to cell surface analytes that can include, without limitation, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction. In some embodiments, the analyte capture agents are capable of binding to cell surface analytes that are post-translationally modified. In such embodiments, analyte capture agents can be specific for cell surface analytes based on a given state of posttranslational modification (e.g., phosphorylation, glycosylation, ubiquitination, nitrosylation, methylation, acetylation or lipidation), such that a cell surface analyte profile can include posttranslational modification information of one or more analytes.

In some embodiments, the analyte capture agent includes a capture agent barcode domain that is conjugated or otherwise attached to the analyte binding moiety. In some embodiments, the capture agent barcode domain is covalently-linked to the analyte binding moiety. In some embodiments, a capture agent barcode domain is a nucleic acid sequence. In some embodiments, a capture agent barcode domain includes an analyte binding moiety barcode and an analyte capture sequence.

As used herein, the term “analyte binding moiety barcode” refers to a barcode that is associated with or otherwise identifies the analyte binding moiety. In some embodiments, by identifying an analyte binding moiety and its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein. For example, an analyte capture agent that is specific to one type of analyte can have coupled thereto a first capture agent barcode domain (e.g., that includes a first analyte binding moiety barcode), while an analyte capture agent that is specific to a different analyte can have a different capture agent barcode domain (e.g., that includes a second barcode analyte binding moiety barcode) coupled thereto. In some aspects, such a capture agent barcode domain can include an analyte binding moiety barcode that permits identification of the analyte binding moiety to which the capture agent barcode domain is coupled. The selection of the capture agent barcode domain can allow significant diversity in terms of sequence, while also being readily attachable to most analyte binding moieties (e.g., antibodies or aptamers) as well as being readily detected, (e.g., using sequencing or array technologies).

In some embodiments, the capture agent barcode domain of an analyte capture agent includes an analyte capture sequence. As used herein, the term “analyte capture sequence” refers to a region or moiety configured to hybridize to, bind to, couple to, or otherwise interact with a capture domain of a capture probe. In some embodiments, an analyte capture sequence includes a nucleic acid sequence that is complementary to or substantially complementary to the capture domain of a capture probe such that the analyte capture sequence hybridizes to the capture domain of the capture probe. In some embodiments, an analyte capture sequence comprises a poly(A) nucleic acid sequence that hybridizes to a capture domain that comprises a poly(T) nucleic acid sequence. In some embodiments, an analyte capture sequence comprises a poly(T) nucleic acid sequence that hybridizes to a capture domain that comprises a poly(A) nucleic acid sequence. In some embodiments, an analyte capture sequence comprises a non-homopolymeric nucleic acid sequence that hybridizes to a capture domain that comprises a non-homopolymeric nucleic acid sequence that is complementary (or substantially complementary) to the non-homopolymeric nucleic acid sequence of the analyte capture region.

In some embodiments of any of the methods described herein, the capture agent barcode domain coupled to the analyte binding moiety includes a cleavable domain. For example, after the analyte capture agent binds to an analyte (e.g., a cell surface analyte), the capture agent barcode domain can be cleaved and collected for downstream analysis according to the methods as described herein.

After the analyte capture sequence hybridizes to the capture domain, it can then interact with padlock probes or snail probes as described below.

### (d) Padlock Probes and Snail Probes

This disclosure features a method for measuring the mislocalization of an analyte in a biological sample using rolling circle amplification (RCA). Initially, after hybridization of an analyte to a capture probe or the complement thereof, in some instances, a padlock probe hybridizes to the extended capture probe, analyte derived molecule, or the complement thereof. As used herein, a “padlock probe” refers to an oligonucleotide that includes, at its 5′ and 3′ ends, sequences (e.g., a first sequence at the 5′ end and a second sequence at the 3′ end) that are complementary to adjacent or nearby portions (e.g., a first portion and a second portion) of the analyte or an analyte derived molecule. Padlock probes are designed such that they comprise multiple substantially or fully complementary sequences to the analyte or analyte derived molecule. As described herein, an analyte includes nucleic acids (e.g., DNA or RNA) in some instances. An example of an analyte derived molecule includes a molecule comprising a protein binding moiety conjugated to an oligonucleotide. A further example is a product of RNA-templated ligation (RTL) as disclosed in US 2021/0348221, US 2021/0285046, and WO 2021/133849, each of which is incorporated by reference in its entirety. Examples and description of analyte derived molecule (also referred to as analyte binding moieties) has been described in WO 2020/176788 and/or U.S. 2020/0277663, each of which is incorporated by reference.

In some instances, Specific Nucleic Acid detection via Intramolecular Ligation (snail) probes are used. SNAIL-RCA (i.e., RCA using snail probes) uses an adaptation of the padlock probe/proximity ligation/rolling circle amplification process and has been shown to amplify the signal from RNA present at relatively low levels of expression. See e.g., O'Huallachain, M. et al. Communications Biology volume 3, Article number: 213 (2020); Nilsson, M. et al. Science. 7522346 (1994); and Wang, X. et al. Science. aat5691 (2018); each of which is incorporated by reference in its entirety. SNAIL-RCA was adopted for directed amplification of RNA in cells by proximity ligation. Typically, two primers are used, one of which binds to its target (e.g., an extended capture probe; a ligation product; or an oligonucleotide from an analyte capture agent; or any complements thereof) at a 3′ position and contains the sequence cognate to a common ligation junction. The second primer contains a sequence proximal, which binds slightly upstream on the target, but which also anneals to the ligation junction. After ligation of the two SNAIL primers, rolling circle amplification occurs which results in amplification of the target or complement thereof.

Upon hybridization of the padlock probe or snail probe to the first and second portions (i.e., sequences) of the analyte or analyte derived molecule, the two ends of the padlock probe are either brought into contact, or an end is extended until the two ends are brought into contact, allowing circularization of the padlock probe or snail probe by ligation (e.g., ligation using any of the methods described herein). The ligation product can be referred to as the “circularized padlock probe” or “circularized snail probe.” After circularization of the padlock probe or snail probe, rolling circle amplification can be used to amplify the circularized padlock probe or snail probe.

In some embodiments, a first sequence of a padlock probe or snail probe includes a sequence that is substantially complementary to a first portion of the analyte or analyte derived molecule. In some embodiments, the first portion of the analyte or analyte derived molecule is 5′ to the second portion of the analyte or the analyte derived molecule. In some embodiments, the first sequence is at least 70% identical (e.g., at least 75% identical, at least 80% identical, at least 85% identical, at least 90% identical, at least 95% identical, or at least 99% identical) to the first portion.

In some embodiments, a backbone sequence of a padlock probe or snail probe includes a sequence that is substantially complementary to an amplification primer. The amplification primer can be a primer used in a rolling circle amplification reaction (RCA) of the ligated padlock probe or snail probe hybridized to the analyte or analyte derived molecules. Rolling circle amplification is well known in the art and includes a process by which circularized nucleic acid molecules are amplified with a DNA polymerase with strand displacement capabilities (and other necessary reagents for amplification to occur), thereby creating multiple concatenated copies of the circularized nucleic acid molecules. In some embodiments, the backbone sequence includes a functional sequence. In some embodiments, the backbone sequence includes a unique molecule identifier (UMI) or barcode sequence (e.g., any of the exemplary barcode sequences described herein). In some embodiments, the barcode sequence includes a sequence that is substantially complementary to an amplification primer. In some instances, the backbone UMI or backbone barcode sequence comprises a sequence (e.g., n=5-50 nucleotides) that is specific to the padlock probe or snail probe. In some instances, the backbone UMI or backbone barcode sequence in the padlock probe or snail probe can be used to identify the padlock or snail sequence.

In some embodiments, a second sequence of a padlock probe or snail probe includes a sequence that is substantially complementary to a second portion of the analyte or analyte derived molecule. In some embodiments, the second portion of the analyte or analyte derived molecule is 3′ to the first portion of the analyte or the analyte derived molecule. In some embodiments, the second sequence is at least 70% identical (e.g., at least 75% identical, at least 80% identical, at least 85% identical, at least 90% identical, at least 95% identical, or at least 99% identical) to the second portion.

In some embodiments, the first sequence is substantially complementary to a first portion of the analyte or analyte derived molecule that is directly adjacent to the second portion of the analyte or analyte derived molecule to which the second sequence is substantially complementary. In such cases, the first sequence is ligated to the second sequence, thereby creating a circularized padlock probe or snail probe.

In some embodiments, the first sequence is substantially complementary to a first portion of the analyte or analyte derived molecule that is not directly adjacent to the second portion of the analyte or analyte derived molecule to which the second sequence is substantially complementary. In such cases, a “gap” exists between where the first sequence is hybridized to the first potion and where the second sequence is hybridized to the second portion. In some embodiments, there is a nucleotide sequence (e.g., a gap) in the analyte between the first portion and the second portion of at least 1-100, 1-90, 1-80, 1-70, 1-60, 1-50, 1-40, 1-30, 1-20, 1-10, 1-9, 1-8, 1-7, 1-6, 1-5, 1-4, 1-3, 1-2 or 1 nucleotide(s). In a non-limiting example, a first sequence having a sequence that is complementary to a sequence 5′ of the gap and a second sequence having a sequence that is complementary to a sequence 3′ of the gap each bind to an analyte leaving a sequence (e.g., the “gap”) in between the first and second sequences that is gap-filled thereby enabling ligation and generation of the circularized padlock probe or snail probe. In some instances, to generate a padlock probe or snail probe that includes a first sequence and a second sequence that are close enough to one another to initiate a ligation step, the second sequence is extended enzymatically (e.g., using a reverse transcriptase).

In some embodiments, the “gap” sequence between the first sequence and the second sequence include one nucleotide, two nucleotides, three nucleotides, four nucleotides, five nucleotides, six nucleotides, seven nucleotides, eight nucleotides, nine nucleotides, ten nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, at least 25 nucleotides, at least 30 nucleotide, at least 35 nucleotides, at least 40 nucleotides, at least 45 nucleotides, or at least 50 nucleotides.

In some embodiments, extending the second sequence of the padlock probe or snail probe includes a nucleic acid extension reaction (e.g., any of the nucleic acid extension reactions described herein). In some embodiments, extending the second sequence of the padlock probe or snail probe includes reverse transcribing the analyte or analyte derived molecule. In some embodiments, extending the second sequence of the padlock probe or snail probe includes using a reverse transcriptase (e.g., any of the reverse transcriptases described herein). In some embodiments, extending the second sequence of the padlock probe or snail probe includes using a Moloney Murine Leukemia Virus (M-MMLV) reverse transcriptase. In some embodiments, the reverse transcriptase includes strand displacement properties. In some embodiments, extending the second sequence of the padlock probe generates a sequence that is complementary to the analyte or the analyte derived molecule. In some embodiments, extending the second sequence of the padlock probe or snail probe generates an extended second sequence of the padlock probe or snail probe that is complementary to the analyte or analyte derived molecule. In some embodiments, second sequence of the padlock probe or snail probe generates a sequence that is adjacent to the first sequence of the padlock probe or snail probe.

In some embodiments, the ligation step includes ligating the second sequence to the first sequence of the padlock probe or snail probe using enzymatic or chemical ligation. In some embodiments where the ligation is chemical ligation, the ligation reaction comprises click chemistry. In some embodiments where the ligation is enzymatic, the ligase is selected from a T4 RNA ligase (Rnl2), a SplintR ligase, a single stranded DNA ligase, or a T4 DNA ligase. In some embodiments, the ligase is a T4 RNA ligase (Rnl2) ligase. In some embodiments, the ligase is a pre-activated T4 DNA ligase as described herein. A non-limiting example describing methods of generating and using pre-activated T4 DNA include U.S. Pat. No. 8,790,873, the entire contents of which are herein incorporated by reference.

### (e) Amplification of Padlock Probes or Snail Probes

This disclosure features a method for determining the mislocalization of an analyte in a biological sample using amplification of a circularized padlock probe or snail probe. In a non-limiting example, the method includes an amplifying step where one or more amplification primers are hybridized to the circularized padlock probe or snail probe and the circularized padlock probe or snail probe is amplified using a polymerase. Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes. The amplification product(s) can be detected by hybridization of detection probes to the amplification product, which identify the location of the mislocalized analyte in the biological sample. The location of the mislocalized analyte can then be compared against an initial (or original) location of an analyte based on imaging prior to permeabilization of the biological sample and migration of the analytes. In some embodiments, the amplifying step includes rolling circle amplification (RCA).

As used herein, rolling circle amplification (RCA) refers to a polymerization reaction carried out using a single-stranded circular DNA (e.g., a circularized padlock probe or snail probe) as a template and an amplification primer that is substantially complementary to the single-stranded circular DNA (e.g., the circularized padlock probe or snail probe) to synthesize multiple continuous single-stranded copies of the template DNA (e.g., the circularized padlock probe or snail probe). In some embodiments, RCA includes hybridizing one or more amplification primers to the circularized padlock probe or snail probe and amplifying the circularized padlock probe or snail probe using a DNA polymerase with strand displacement activity, for example Phi29 DNA polymerase. In some embodiments, a first RCA reaction includes a first padlock probe or snail probe and a first amplification primer (or plurality of first amplification primers). A first RCA reaction can include the first padlock probe or snail probe hybridizing to a first analyte or first analyte derived molecule.

In some embodiments, an RCA reaction is carried out using a DNA polymerase and a dNTP mix including uracil, adenine, guanine and cytosine. In such cases, the uracils are incorporated into the amplified padlock probe or snail probe. In some embodiments, an RCA reaction is carried out using a DNA polymerase and a dNTP mix including uracil, adenine, guanine, cytosine and thymine. In such cases, uracils and thymines are both incorporated into the amplified padlock probe or snail probe.

In some instances, the dNTP molecule is labeled. In some instances, the label is a fluorescent label that is chemically coupled to the nucleotide. A fluorescent label can be attached to any of the following nucleotides dATP, dCTP, dGTP, dUTP, and dTTP. The fluorescent label can be any fluorescent label known in the art, including but not limited to cyanine 5 dye (Cy-5), cyanine 3 dye (Cy-3), fluorescein, rhodamine, phosphor, coumarin, polymethadine dye, fluorescent phosphoramidite, Texas red, green fluorescent protein, acridine, cyanine, and 5-(2′-aminoethyl)-aminonaphthalene-1-sulfonic acid (EDANS).

In some embodiments, the method further includes a second RCA reaction. For example, following a first RCA reaction, a second padlock probe or snail probe and a second amplification primer are added to the substrate. In some embodiments, the second padlock probe or snail probe hybridizes to a second analyte or second analyte derived molecule and is ligated thereby creating a second circularized padlock probe or snail probe. The second circularized padlock probe or snail probe can then be amplified using RCA.

In some embodiments, the second padlock probes or snail probes include a third sequence and a fourth sequence where each are substantially complementary to sequences on a second analyte (e.g., a different analyte then was bound by the first padlock probe or snail probe in the first RCA reaction). In some embodiments, the second padlock probe or snail probe includes: (i) a third sequence substantially complementary to the second analyte or second analyte derived molecule, (ii) a backbone sequence comprising a barcode, and (iii) a fourth sequence that is substantially complementary to a sequence adjacent to the third sequence in the second analyte or second analyte derived molecule. In some embodiments, the third sequence is at least 70% identical (e.g., at least 75% identical, at least 80% identical, at least 85% identical, at least 90% identical, at least 95% identical, or at least 99% identical) to a portion of the second analyte or second analyte derived molecule. In some embodiments, the fourth sequence is at least 70% identical (e.g., at least 75% identical, at least 80% identical, at least 85% identical, at least 90% identical, at least 95% identical, or at least 99% identical) to a sequence adjacent to the third sequence in the second analyte or second analyte derived molecule.

In some embodiments, the method further includes digesting the amplified circularized padlock probe or snail probe by using an enzyme that cleaves a nucleic acid sequence (e.g., an enzyme that cleaves nucleic acid sequences that include uracil). Digesting and/or removing the amplified circularized padlock probe or snail probe after detection of the detection probes can alleviate crowding that may result from the physical size of the RCA product and/or the detection probe. In some embodiments, the enzymatic digestion of the amplified circularized padlock probe or snail probe includes a uracil-DNA glycosylase (UDG) that cleaves nucleic acid sequences that include uracil incorporated into the amplified padlock probe or snail probe. In some embodiments, the enzyme is a combination of enzymes, such as UDG and DNA glycosylase lyase such as Endonuclease VIII.

In some embodiments, an amplification primer includes a sequence that is substantially complementary to one or more of the first sequence, the backbone sequence, or the second sequence of the padlock probe or snail probe. For example, the amplification primer can be substantially complementary to the backbone sequence. In some embodiments, the amplification primer includes a sequence that is substantially complementary to the padlock probe or snail probe and an additional portion of the analyte or analyte derived molecule. For example, the amplification primer can be substantially complementary to the backbone sequence and a portion of the analyte or analyte derived molecule that does not include the first and second portion of the padlock probe or snail probe. In some embodiments, the amplification primer includes a sequence that is substantially complementary to two or more of the first sequence, the backbone sequence, or the second sequence of the padlock probe or snail probe and an additional portion of the analyte or analyte derived molecule. By substantially complementary, it is meant that the amplification primer is at least 70%, at least 75%, at least 80%, at least 85%, at least 90%, at least 91%, at least 92%, at least 93%, at least 94%, at least 95%, at least 96%, at least 97%, at least 98%, at least 99%, complementary to a sequence in the circularized padlock probe or snail probe. By substantially complementary, it is meant that the amplification primer is at least 70%, at least 75%, at least 80%, at least 85%, at least 90%, at least 91%, at least 92%, at least 93%, at least 94%, at least 95%, at least 96%, at least 97%, at least 98%, at least 99% complementary to a sequence in the analyte or analyte derived molecule.

### (f) Detection Probes and Signal Detection

This disclosure features a method for determining the mislocalization of an analyte in a biological sample using detection of a signal that corresponds to an amplified circularized padlock probe or snail probe (which thereby corresponds to an analyte or analyte binding molecule). In some embodiments, the method includes a step of detecting a signal corresponding to the amplified circularized padlock probe or snail probe on the substrate, thereby identifying the location of the mislocalized analyte in the biological sample. In some embodiments, the method includes a detecting step that includes determining (i) all or a part of the sequence of the amplified circularized padlock probe or snail probe on the substrate and using the determined sequence of the padlock probe or snail probe to identify the location of the analyte in the biological sample.

In some embodiments, the detecting step includes contacting the amplified circularized padlock probe or snail probe with a plurality of detection probes. In some embodiments, a detection probe of the plurality of detection probes includes a sequence that is substantially complementary to a sequence of the padlock probe or snail probe, circularized padlock probe or snail probe, or amplified circularized padlock probe or snail probe and a detectable label. For example, the detection probe of the plurality of detection probes can include a sequence that is substantially complementary to a sequence of amplified circularized padlock probe or snail probe and a detectable label. By substantially complementary, it is meant that the detection probe is at least 70%, at least 75%, at least 80%, at least 85%, at least 90%, at least 91%, at least 92%, at least 93%, at least 94%, at least 95%, at least 96%, at least 97%, at least 98%, at least 99% complementary to a sequence in the amplified circularized padlock probe or snail probe.

In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350 (350 N-Hydroxysuccinimide ester, a fluorophore with an excitation wavelength of about 350 nm), Alexa Fluor® 430 (430 N-Hydroxysuccinimide ester, a fluorophore with an excitation wavelength of about 430 nm), Alexa Fluor® 488 (488 N-Hydroxysuccinimide ester, a fluorophore with an excitation wavelength of about 488 nm), Alexa Fluor® 532 (532 N-Hydroxysuccinimide ester, a fluorophore with an excitation wavelength of about 532 nm), Alexa Fluor® 546 (546 N-Hydroxysuccinimide ester, a fluorophore with an excitation wavelength of about 546 nm), Alexa Fluor® 555 (555 N-Hydroxysuccinimide ester, a fluorophore with an excitation wavelength of about 555 nm), Alexa Fluor® 568 (568 N-Hydroxysuccinimide ester, a fluorophore with an excitation wavelength of about 568 nm), Alexa Fluor® 594 (594 N-Hydroxysuccinimide ester, a fluorophore with an excitation wavelength of about 594 nm), Alexa Fluor® 633 (633 N-Hydroxysuccinimide ester, a fluorophore with an excitation wavelength of about 633 nm), Alexa Fluor® 647 (647 N-Hydroxysuccinimide ester, a fluorophore with an excitation wavelength of about 647 nm), Alexa Fluor® 660 (660 N-Hydroxysuccinimide ester, a fluorophore with an excitation wavelength of about 660 nm), Alexa Fluor® 680 (680 N-Hydroxysuccinimide ester, a fluorophore with an excitation wavelength of about 680 nm), Alexa Fluor® 700 (700 N-Hydroxysuccinimide ester, a fluorophore with an excitation wavelength of about 700 nm), Alexa Fluor® 750 (750 N-Hydroxysuccinimide ester, a fluorophore with an excitation wavelength of about 750 nm), Allophycocyanin (APC), AMCA I AMCA-X, 7-Amino-4-methylcoumarin, 6-Aminoquinoline, Aniline Blue, ANS (1-anilinonaphthalene-8-sulfonate), APC-Cy™ 7 (allophycocyanin-Cyanine 7), ATTO-TAG™ CBQCA (3-(4-carboxybenzoyl)quinoline-2-carboxaldehyde), ATTO-TAG™ FQ (3-(2-furoyl)quinoline-2-carboxaldehyde), Auramine O-Feulgen, BCECF (2′,7′-bis-(2-carboxyethyl)-5-(and-6)-carboxyfluorescein) (high pH), BFP (Blue Fluorescent Protein), BFP/GFP (Green Fluorescent Protein) FRET (Fluorescence Resonance Energy Transfer), BOBO™-1/BO-PRO™-1 (a blue-emitting fluorescent nucleic acid stain with an excitation wavelength of about 462 nm), BOBO™-3/BO-PRO™-3 (nucleic acid stain with an excitation wavelength of about 470 nm), BODIPY® FL (borondipyrromethene dye for fluorescein channel), BODIPY® TMR (borondipyrromethene dye for tetramethylrhodamine channel), BODIPY® TR-X (borondipyrromethene dye for the Texas Red® (sulforhodamine 101 acid chloride) channel with a succinimidyl ester modification), BODIPY® 530/550 (530/550 N-Hydroxysuccinimide ester, borondipyrromethene dye with an excitation wavelength_of about 530 nm), BODIPY® 558/568 (558/568 N-Hydroxysuccinimide ester, borondipyrromethene dye with an excitation wavelength of about 558 nm), BODIPY® 564/570 (564/570 N-Hydroxysuccinimide ester, borondipyrromethene dye with an excitation wavelength of about 564 nm), BODIPY® 581/591 (581/591 N-Hydroxysuccinimide ester, borondipyrromethene dye with an excitation wavelength of about 581 nm), BODIPY® 630/650-X (630/650-X N-Hydroxysuccinimide ester, borondipyrromethene dye with an excitation wavelength of about 630 nm and a succinimidyl ester modification), BODIPY® 650-665-X (650/660-X N-Hydroxysuccinimide ester, borondipyrromethene dye with an excitation wavelength of about 650 nm and a succinimidyl ester modification), BTC (bromothenylcytosine), Calcein (Fluorescein-bis(methyliminodiacetic acid), Calcein Blue (4-Methylumbelliferone-8-methyliminodiacetic acid), Calcium Crimson™ (5-{[4-(bis{2-[(acetyloxy)methoxy]-2-oxoethyl}amino)-3-{2-[2-(bis{2-[(acetyloxy)methoxy]-2-oxoethyl}amino)phenoxy]ethoxy}phenyl]sulfamoyl}-2-(2,3,6,7,12,13,16,17-octahydro-1H,5H,11H,15H-pyrido[3,2,1-ij]quinolizino[1′,9′:6,7,8]chromeno[2,3-f]quinolin-4-ium-9-yl)benzenesulfonate, a cell-permeant light-excitable Ca⁺² indicator), Calcium Green-I™ (2,2′-((2-(2-(2-(bis(carboxylatomethyl)amino)-5-(3-carboxylato-4-(2,7-dichloro-6-oxido-3-oxo-3H-xanthen-9-yl)benzamido)phenoxy)ethoxy)phenyl)azanediyl)diacetate, a cell-permeant light-excitable Ca⁺² indicator), Calcium Orange™ (9-[4-[[[[4-[bis[2-[(acetyloxy)methoxy]-2-oxoethyl]amino]-3-[2-[2-[bis[2-[(acetyloxy)methoxy]-2-oxoethyl]amino]phenoxy]ethoxy]phenyl]amino]thioxomethyl]amino]-2-carboxyphenyl]-3,6-bis(dimethylamino), a cell-permeant light-excitable Ca⁺² indicator), Calcofluor® White (4,4′-bis((2-anilino-4-(bis(2-hydroxyethyl)amino)-1,3,5-triazin-6-yl)amino)stilbene-2,2′-disulfonic acid, a fluorescent polysaccharide indicator), 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue® (N-(4-{[4-(diethylamino)phenyl][4-(ethylamino)-2-naphthyl]methylene}cyclohexa-2,5-dien-1-ylidene)-N-ethylethanaminium, a sulfonated pyrene dye with an excitation wavelength of about 396 nm), Cascade Yellow™ (5-{2-[1-(3-{[(2,5-dioxopyrrolidin-1-yl)oxy]carbonyl}benzyl)pyridinium-4-yl]-1,3-oxazol-5-yl}-2-methoxybenzenesulfonate, a sulfonated pyrene dye with an excitation wavelength of about 402 nm), CCF2 (Coumarin cephalosporin fluorescein), GeneBLAzer™ kit using a β-lactamase substrate), CFP (Cyan Fluorescent Protein), CFP/YFP FRET, Chromomycin A3, ClNERF (low pH), CPM (7-Diethylamino-3-(4′-Maleimidylphenyl)-4-Methylcoumarin), 6-CR (6-Carboxyrhodamine) 6G, CTC (5-Cyano-2,3-ditolyl tetrazolium chloride) Formazan (emission peak, 630 nm), Cy2® (Cyanine2 N-Hydroxysuccinimide ester, tetramethylindo(di)-carbocyanine dye with excitation wavelength of about 490 nm), Cy3® (Cyanine3, tetramethylindo(di)-carbocyanine dye with excitation wavelength of about 555 nm), Cy3.5® (Cyanine3.5, tetramethylindo(di)-carbocyanine dye with excitation wavelength of about 591 nm), Cy5® (Cyanine5, tetramethylindo(di)-carbocyanine dye with excitation wavelength of about 646 nm), Cy5.5® (Cyanine5.5, tetramethylindo(di)-carbocyanine dye with excitation wavelength of about 675 nm), Cy7® (Cyanine7, tetramethylindo(di)-carbocyanine dye with excitation wavelength of about 743 nm), Cychrome (PE-Cy5®), Dansyl amine, Dansyl cadaverine, Dansyl chloride, DAPI (4′,6-diamidino-2-phenylindole), Dapoxyl (2-aminoethyl)sulfonamide, DCFH (Diacetyldichlorofluorescein), DHR (Dihydrorhodamine), DiA (4-Di-16-ASP), DiD (DilC18(5)), DIDS (DiIC18(5); 1,1′-dioctadecyl-3,3,3′,3′-tetramethylindodicarbocyanine, 4-chlorobenzenesulfonate), Dil (1,1′-Dioctadecyl-3,3,3′,3′-Tetramethylindocarbocyanine Perchlorate; DilC₁₈(3)), DiO (DiOC₁₈(3)), DiR (DilC₁₈(7)), Di-4 ANEPPS (AminoNaphthylEthenylPyridinium), Di-8 ANEPPS, DM-NERF (4.5-6.5 pH), DsRed (Red Fluorescent Protein), EBFP (Enhanced Blue Fluorescent Protein), ECFP (Enhanced Cyan Fluorescent Protein), EGFP (Enhanced Green Fluorescent Protein), ELF®-97 alcohol (a phosphatase substrate), Eosin, Erythrosin, Ethidium bromide, Ethidium homodimer-1 (EthD-1), Europium (III) Chloride, 5-FAM (5-Carboxyfluorescein), Fast Blue (4-Benzoylamino-2,5-dimethoxyaniline), Fluorescein-dT phosphoramidite, FITC (Fluorescein isothiocyanate), Fluo-3, Fluo-4, FluorX® (carboxyfluorescein derivative), Fluoro-Gold™ (Hydroxystilbamidine, high pH), Fluoro-Gold™ (Hydroxystilbamidine, low pH), Fluoro-Jade, FM® 1-43 (neuron-specific fluorochrome), Fura-2 (high calcium), Fura-2/BCECF (2′,7′-Bis-(2-Carboxyethyl)-5-(and-6), Fura Red™ (fura-2 analog, high calcium), Fura Red™ I Fluo-3 (fura-2 analog), GeneBLAzer™ (CCF2 β-lactamase substrate), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS (N-(Iodoacetaminoethyl)-1-naphthylamine-5-sulfonic acid), Indo-1 (high calcium), Indo-1 (low calcium), Indodicarbocyanine, Indotricarbocyanine, JC-I (5,5′,6,6′-tetrachloro-1,1′,3,3′-tetraethylbenzimidazolylcarbocyanine iodide), 6-JOE (6-Carboxy-4′,5′-Dichloro-2′,7′-Dimethoxyfluorescein), JOJO™-1 I JO-PRO™-1 (nucleic acid stain with an excitation wavelength of about 532 nm), LDS 751 (+DNA), LDS 751 (+RNA), LOLO™-1/LO-PRO™-1 (nucleic acid stain with an excitation wavelength of about 565 nm), Lucifer Yellow (6-Amino-2,3-dihydro-1,3-dioxo-2-hydrazinocarbonylamino-1H-benz[d,e]isoquinoline-5,8-disulfonic acid dilithium salt), LysoSensor™ Blue (pH-sensitive ratiometric probe, pH 5), LysoSensor™ Green (pH-sensitive ratiometric probe, pH 5), LysoSensor™ Yellow/Blue (pH-sensitive ratiometric probe, pH 4.2), LysoTracker® Green (fluorophore linked to a weak base), LysoTracker® Red (fluorophore linked to a weak base), LysoTracker® Yellow (fluorophore linked to a weak base), Furaptra (MagFura-2), Mag-Indo-1 (2-(4-Amino-3-hydroxyphenyl)indole-6-carboxylic acid N′,N′,O′-triacetic acid tetrapotassium salt, Indo 1 analog tetrapotassium salt), Magnesium Green™ (fluorescent magnesium indicator), Marina Blue® (1,2-Dihexadecanoyl-sn-Glycero-3-Phosphoethanolamine, fluorescent carbohydrazide dye), 4-Methylumbelliferone, Mithramycin, MitoTracker® Green (mitochondria-specific green-fluorescent stain), MitoTracker® Orange (mitochondria-specific orange-fluorescent stain), MitoTracker® Red (mitochondria-specific red-fluorescent stain), NBD (amine), Nile Red, Oregon Green® 488 (green-fluorescent dye with an excitation wavelength of about 488 nm), Oregon Green® 500 (green-fluorescent dye with an excitation wavelength of about 500 nm), Oregon Green® 514 (green-fluorescent dye with an excitation wavelength of about 506 nm), Pacific Blue (3-carboxy-6,8-difluoro-7-hydroxycoumarin), PBFI (Potassium-binding Benzofuran Isophthalate Acetoxymethyl ester), PE (Rphycoerythrin), PE-Cy5, PE-Cy7, PE-Texas Red®, PerCP (Peridinin chlorphyll protein), PerCP-Cy5.5 (Peridinin chlorophyll protein-Cyanine5.5, TruRed), PharRed (APC-Cy7), C-phycocyanin, R-phycocyanin, R-phycoerythrin (PE), PI (Propidium Iodide), PKH26, PKH67, POPO™-1/PO-PRO™-1 (high-affinity carbocyanine dimeric nucleic acid stain), POPO™-3/PO-PRO™-3 (high-affinity carbocyanine dimeric nucleic acid stain), PyMPO (1-(3-carboxybenzyl)-4-(5-(4-methoxyphenyl)oxazol-2-yl)pyridinium bromide), Pyrene, Pyronin Y, Quantam Red (PE-Cy5), Quinacrine mustard, R670 (PE-Cy5), Red 613 (PE-Texas Red®), Red Fluorescent Protein (DsRed), Resorufin, RH 414, Rhod-2, Rhodamine B, Rhodamine Green™ (triarylmethane dye), Rhodamine Red™ (triarylmethane dye), Rhodamine Phalloidin, Rhodamine 110, Rhodamine 123, 5-ROX (5-carboxy-X-rhodamine), S65A, S65C, 5 S65L, S65T, SBFI (Sodium-binding benzofuran isophthalate), SITS (Stilbene isothiosulphonic acid), SNAFL®-1 (5′ (and 6′)-carboxyseminaphthofluorescein, high pH), SNAFL®-2 (carboxy seminaphthofluorescein), SNARF®-1 (pH-dependent fluorescent dye, high pH), SNARF®-1 (pH-dependent fluorescent dye, low pH), Sodium Green™ (tetra (tetramethylammonium) salt, fluorescent sodium indicator), SpectrumAqua® (fluorophore with excitation wavelength of about 433 nm), SpectrumGreen™ #1 (fluorophore with excitation wavelength of about 497 nm), SpectrumGreen™ #2 (fluorophore with excitation wavelength of about 509 nm), SpectrumOrange™ (fluorophore with excitation wavelength of about 559 nm), SpectrumRed™ (fluorophore with excitation wavelength of about 587 nm), SYTOR 11 (fluorophore with excitation wavelength of about 508 nm), SYTOR 13 (fluorophore with excitation wavelength of about 488 nm), SYTO® 17 (fluorophore with excitation wavelength of about 621 nm), SYTOR 45 (fluorophore with excitation wavelength of about 452 nm), SYTOX® Blue (fluorophore with excitation wavelength of about 445 nm), SYTOX® Green (fluorophore with excitation wavelength of about 504 nm), SYTOX® Orange (fluorophore with excitation wavelength of about 547 nm), 5-TAMRA (5-Carboxytetramethylrhodamine), Tetramethylrhodamine (TRITC), Texas Red® I Texas Red®-X (fluorophore with excitation wavelength of about 595 nm), Texas Red®-X (fluorophore with excitation wavelength of about 595 nm and N-Hydroxysuccinimide Ester modification), Thiadicarbocyanine, Thiazole Orange, TOTO®-1/TO-PRO®-1 (thiazole orange homodimer nucleic acid stain), TOTO®-3 I TO-PRO®-3 (thiazole red homodimer nucleic acid stain), TO-PRO®-5 (carbocyanine-based nucleic acid stain), Tri-color (PE-Cy5), WW 781 ([3-methyl-1-p-sulfophenyl-5-pyrazolone-(4)]-[1,3-dibutylbarbit uric acid]-pentamethine oxonol), X-Rhodamine (XRITC), Y66F, Y66H, Y66W, YFP (Yellow Fluorescent Protein), YOYO®-1/YO-PRO®-1 (monomethine cyanine nucleic acid stain), YOYO®-3/YO-PRO®-3 (monomethine cyanine nucleic acid stain), 6-FAM (Fluorescein), 6-FAM (N-Hydroxysuccinimide ester), 6-FAM (Azide), HEX (Hexachlorofluorescein), TAMRA (Tetramethylrhodamine, N-Hydroxysuccinimide Ester), Yakima Yellow®, MAX, TET (Tetrachloro fluorescein), TEX615, ATTO 488, ATTO 532, ATTO 550, ATTO 565, ATTO Rhol01, ATTO 590, ATTO 633, ATTO 647N, TYE 563, TYE 665, TYE 705, 5′ IRDye® 700 (infrared fluorescent nucleic acid stain), 5′ IRDye® 800 (infrared fluorescent nucleic acid stain), 5′ IRDye® 800CW (infrared fluorescent nucleic acid stain with an N-Hydroxysuccinimide ester modification), WellRED D4 Dye (cyanine-based near-infrared dye), WellRED D3 Dye (cyanine-based near-infrared dye), WellRED D2 Dye (cyanine-based near-infrared dye), Lightcycler® 640 (red fluorescent dye with an N-Hydroxysuccinimide ester modification), and Dy 750 (betainic dye with an N-Hydroxysuccinimide ester modification).

In some embodiments, a detection probe of the plurality of detection probes includes a nucleotide sequence that is different from other detection probes, thereby enabling detection of signals from two or more detection probe sequences (e.g., two or more different amplified circularized padlock probe or snail probe). In some embodiments, each of the two or more different nucleotide sequences is located on the same amplified circularized padlock probe or snail probe. In some embodiments, each of the two or more different sequences is located on two or more different amplified circularized padlock probe or snail probes (e.g., each amplified circularized padlock probe or snail probe is derived from a different analyte or analyte derived molecule).

In some embodiments, the detecting step includes contacting the amplified circularized padlock probe or snail probe with a detectable label that can hybridize to nucleic acid sequences in a non-sequence dependent manner. In such cases, the single-stranded copies of the template DNA produced by RCA can be detected using fluorescent dyes that non-specifically bind to the single-stranded nucleic acid (e.g., the amplified circularized padlock probe or snail probe). For example, in the case where an analyte is an amplified circularized padlock probe or snail probe, a fluorescent dye can bind, either directly or indirectly, to the single stranded nucleic acid of the RCA product. The dye can then be detected as a signal corresponding to the amplified circularized padlock probe or snail probe (i.e., the location and/or the abundance of the circularized padlock probe or snail probe). Non-limiting examples of fluorescent dyes that can bind to single stranded nucleic acids include: TOTO®-1/TO-PRO®-1, TOTO®-3/TO-PRO®-3, TO-PRO®-5, Ethidium bromide, Ethidium homodimer-1 (EthD-1), YOYO®-1/YO-PRO®-1, YOYO®-3/YO-PRO®-3, 7-AAD (7-Aminoactinomycin D), and OliGreen®.

In some embodiments, the detecting step includes contacting the amplified circularized padlock probe or snail probe with a detection probe (e.g., any of the exemplary detection probes described herein (e.g., a detection probe that includes one or more fluorophores)) and a fluorescent dye that can label single-stranded DNA in a non-sequence dependent manner (e.g., any of the exemplary fluorescent dyes described herein). In such cases, the fluorescent dye can be used to normalize the fluorescent signal detected from the detection probe.

In some embodiments, detecting the signal or signals that correspond to the amplified circularized padlock probes or snail probes on the substrate include obtaining an image corresponding to the analyte and/or analyte derived molecule on the substrate. In some embodiments, the method further includes registering image coordinates to a fiducial marker.

In some embodiments, the method includes repeating the detecting step with a second plurality of detection probes. In such cases, the method includes removing the detection probes from the first detecting step (e.g., via enzymatic digestion) and contacting the amplified circularized padlock probe or snail probe with a second plurality of detection probes. A detection probe of the second plurality of detection probes includes a sequence that is substantially complementary to a sequence of the padlock probe or snail probe and that is non-overlapping, partially overlapping, or completely overlapping with the sequence to which a detection probe from the first plurality of detection probes is substantially complementary.

In some embodiments, determining (i) all or a part of the sequence of the amplified circularized padlock probe or snail probe on the substrate includes determining the barcode sequence on the padlock probe or snail probe, wherein the location of the analyte in the biological sample can be identified. In situ sequencing of the analyte or analyte derived molecule, the nucleic acid molecule, the analyte binding moiety barcode, or the ligation product process is described in PCT Patent Application Publication No. WO 2020/047005, which is incorporated by reference in its entirety.

### (g) Slides, Biological Samples, Analytes, and Preparation of the Same

### (i) Slides, Biological Samples, and Analytes

This disclosure features a method for identifying a location of an analyte in a biological sample using a substrate that includes a plurality of capture probes, where a capture probe of the plurality of capture probes include a capture domain. In some embodiments, the capture probe is affixed to the substrate at a 5′ end. In some embodiments, the plurality of capture probes are uniformly distributed on a surface of the substrate, following no particular pattern. In some embodiments, the plurality of capture probes are located on a surface of the substrate but are distributed on the substrate according to a pattern. In some instances, the capture probes do not include spatial barcodes. Instead, to determine mislocalization of an analyte, spatial analysis is performed with the aid of fluorescent readout, as described herein. Thus, in some instances, the substrate comprises a lawn of capture probes that include a capture domain, but do not include a spatial barcode.

In other embodiments, the substrate includes a plurality of capture probes, where a capture probe of the plurality of capture probes includes a capture domain and a spatial barcode. In some embodiments, the substrate includes a fiducial marker (e.g., any of the fiducial markers described herein). The fiducial markers can be used as a point of reference on the substrate. For example, the spatial location of a signal detected from a detection probe can be determined by using the fiducial marker as a point of reference on the substrate. In some cases, the fiducial marker can be present on a substrate to aid in orientation of the biological sample. In some embodiments, a fiducial marker can be an immobilized molecule with which a detectable signal molecule (e.g., a detectable label from a detection probe) can interact to generate a signal. For example, a fiducial marker can comprise a nucleic acid linked or coupled to a chemical moiety capable of fluorescing when subjected to light of a specific wavelength (or range of wavelengths) at a fixed position on the substrate.

In some embodiments, the biological sample comprises a tissue section. In some embodiments, the biological sample comprises a fresh frozen sample. In some embodiments, the biological sample comprises live or fixed single cells. In some embodiments, the biological sample comprises a FFPE sample.

In some embodiments, the method includes obtaining an image of the biological sample; registering the image data to spatial locations on an array. In some embodiments, the imaging includes a brightfield image. In some embodiments, the method further includes contacting the biological sample with one or more stains. In some embodiments, the one or more stains comprise a histology stain (e.g., any of the histology stains described herein or known in the art). In some embodiments, the one or more stains comprises hematoxylin and/or eosin. In some embodiments, the one or more stains comprise one or more optical labels (e.g., any of the optical labels described herein). In some embodiments, the one or more optical labels are selected from the group consisting of: fluorescent, radioactive, chemiluminescent, calorimetric, or colorimetric labels.

In some embodiments, the methods are applied to analyte, or analyte derived molecules (e.g., an mRNA molecule, a gDNA molecule, a product of reverse transcription (e.g., an extended capture probe), and an analyte binding moiety barcode (e.g., a binding moiety barcode that identifies that analyte binding moiety (e.g., an antibody))). In some embodiments, the analyte or analyte derived molecules comprise RNA and/or DNA. In some embodiments, the analyte or analyte derived molecules comprise one or more proteins.

### (ii) Imaging and Staining

In some instances, biological samples can be stained using a wide variety of stains and staining techniques. In some instances, the biological sample is stained before adding the probes to the biological sample. In some instances, the biological sample is stained after adding the probes to the biological sample.

In some instances, the biological sample is a section of a tissue (e.g., a 10 μm section). In some instances, the biological sample is dried after placement onto a glass slide. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or decoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are probed using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

(iii) Preparation of Sample for Application of Probes

In some instances, additional reagents are added to the biological sample, prior to the addition of the probes. Additional reagents can be any reagent known in the art, so long as it preserves the integrity of an analyte.

In some instances, the biological sample is deparaffinized. Deparaffinization can be achieved using any method known in the art. For example, in some instances, the biological sample is treated with a series of washes that include xylene and various concentrations of ethanol. In some instances, methods of deparaffinization include treatment with xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization.

In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for probe application include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some embodiments of any of the methods for identifying a location of an analyte in a biological sample, the method includes permeabilizing (e.g., using any of the exemplary permeabilization methods described herein) the biological sample before, contemporaneously with, or after exposing the biological sample to the capture probe. In some embodiments of any of the methods for identifying a location of an analyte in a biological sample, the method includes processing the biological sample using any of the methods described herein. For example, the method can include selecting a region of interest of the biological sample by laser-capture microdissection prior to exposing the biological sample to the capture probe.

In some instances, the methods of preparing a biological sample for probe application include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

### (h) Substrates and Capture Probes

This disclosure features a method of identifying a location of an analyte in a biological sample using a substrate that includes a plurality of capture probes, where a capture probe of the plurality of capture probes includes a capture domain. In some embodiments, the capture probe does not include a spatial barcode. In some embodiments, the capture probe includes a spatial barcode.

In some embodiments, a capture probe of the plurality of capture probes is affixed to the substrate at the 5′ end of the capture probe. In such cases, in order to prevent extension of the capture probe during the amplifying step, the capture probe includes a blocking moiety on the 3′ end. Blocking or modifying the capture probes, particularly at the free 3′ end of the capture domain, prior to contacting the biological sample with the array, prevents (1) modification of the capture probes, e.g., prevents the addition of a poly(A) tail to the free 3′ end of the capture probes and (2) extension of the capture probe during amplification of the circularized padlock probe or snail probe.

In some embodiments, the ability to prevent capture probe extension is temporary (e.g., blocking extension of the capture probe during the amplifying step but allowing extension during, for example, a step that purposely extends the capture probe). In such cases, the blocking moiety on the 3′ end can be removed and the capture probe can be extended. For example, the method can include a reversible blocking moiety on the 3′ end of the capture probe in a blocking position during amplification but in a non-blocking position (e.g., removed by cleavage) during a capture probe extension step.

In some embodiments where a capture probe of the plurality of capture probes is attached to the substrate at the 5′ end, the capture probe is blocked at the 3′ end. In some embodiments, the capture probes can be reversibly masked or modified such that the capture domain of the capture probe does not include a free 3′ end. In some embodiments, the 3′ end is removed, modified, or made inaccessible so that the capture domain is not susceptible to the process used to modify the nucleic acid of the biological sample, e.g., ligation or extension. A capture probe can be blocked at the 3′ end by a blocking moiety. The blocking moiety can be a reversible blocking moiety. In some cases, the 3′ end of the capture probe includes a removable hairpin. In some cases, the 3′ end includes a 3′ blocking group present on the sugar moiety of the nucleotide at the 3′ end. In such cases, the 3′ blocking group can be chemically removed from the nucleic acid.

In some embodiments, the free 3′ end of the capture domain can be blocked by chemical modification, e.g., addition of an azidomethyl group as a chemically reversible capping moiety such that the capture probes do not include a free 3′ end. Non-limiting examples of 3′ modifications include dideoxy C-3′ (3′-ddC), 3′ inverted dT, 3′ C3 spacer, 3′ Amino, and 3′ phosphorylation.

In some embodiments where a capture probe of the plurality of capture probes is attached to the substrate at the 5′ end and the 3′ is blocked, additional nucleotides are added to the 3′ end in order to reveal and/or restore the blocked 3′ end of the capture probe, thereby creating a free 3′ end.

In some embodiments, the capture domain includes a sequence that is at least partially complementary to the analyte or the analyte derived molecule. In some embodiments, the capture domain of the capture probe includes a poly-dT sequence. In some embodiments, the capture domain of the capture probe includes a sequence complementary to an analyte of interest (e.g., Secretoglobin Family 1A Member 1 (scgb1a1) or hippocalcin (HPCA),).

In some embodiments, the capture domain includes a functional domain. In some embodiments, the functional domain includes a primer sequence. In some embodiments, the capture probe includes a cleavage domain. In some embodiments, the cleavage domain includes a cleavable linker from the group consisting of a photocleavable linker, a UV-cleavable linker, an enzyme-cleavable linker, or a pH-sensitive cleavable linker.

In some embodiments, the capture domain of the capture probe comprises a non-homopolymeric sequence. In some embodiments, the capture domain is a defined sequence. In such cases, the defined sequence can be substantially complementary to a portion of the amplified padlock probe or snail probe. When the capture probe is substantially complementary to a portion of the amplified oligonucleotide, the capture domain can be used to capture all or a portion of the padlock probe or snail probe. In some instances, a defined sequence is about 5 to 50 nucleotides in length (e.g., about 5 to 25, about 5 to 20, about 10 to 25, about 10 to 20). In some instances, the length of the defined sequence is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides long. Defined sequences can include natural or synthetic nucleic acids. It is appreciated that a skilled artisan can design the defined sequence in order to capture a particular target or particular targets of interest.

In preferred embodiments the capture probe is blocked on the 3′ end using any blocking method as previously described. The analyte or analyte derived molecule is captured by the capture domain of the capture probe, padlock probes or snail probes are hybridized and circularized and RCA performed, and detection of the analyte or analyte derived molecule determined via one or more detection probes. In this particular embodiment, a blocked capture probe mitigates any modification or extension of the capture probe during amplification of the circularized padlock probe or snail probe.

### (i) Methods of Performing RNA Analysis on a Related Biological Sample

In some embodiments, after determining the mislocalization of analytes in a biological sample, RNA analysis can be performed on a related biological sample (e.g., a serial biological section) using a spatial array. In some instances, the related biological sample, for example a serial section taken from a tissue block that was used to determine mislocalization of analytes, is placed on a second substrate and analytes are captured using methods disclosed herein. The methods include determining abundance and/or location of a plurality of analytes in a related biological sample. In some examples, determining the abundance and/or location includes: (a) contacting a second spatial array comprising a comprising a plurality of second spatial capture probes with the related (e.g., serial) biological section, wherein a second spatial capture probe of the plurality of second spatial capture probes comprises a second spatial barcode and a second capture domain, (b) hybridizing an analyte from the related (e.g., serial) biological section to a second capture domain, and (c) determining: (i) all or a part of a sequence of the analyte from the related (e.g., serial) biological section, or a complement thereof, and (ii) the sequence of the second spatial barcode, or a complement thereof, and using the determined sequence of (i) and (ii) to determine the abundance and/or the location of the analyte from the serial biological section. The methods of RNA analysis include preparation of the related biological sample, which includes but is not limited to sectioning the sample, staining and deparaffinizing the sample, imaging the sample, and permeabilizing the sample. In some instances, permeabilization includes treatment with a solution comprising proteinase K, pepsin, or a combination of both. Preparation of biological samples is disclosed in WO 2020/176788 and/or U.S. Patent Application Publication No. 2020/0277663, each of which is incorporated by reference in its entirety.

In some instances, analytes are captured by capture probes on the second spatial array. After an analyte from the sample has hybridized or otherwise been associated with a capture probe according to any of the methods described above in connection with the general spatial cell-based analytical methodology, the barcoded constructs that result from hybridization/association are analyzed.

In some embodiments, after contacting a biological sample with a substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove all or at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture probe bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with an array comprising a plurality of capture probes, wherein a capture probe of the plurality captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting a region of interest in the biological sample to spatial transcriptomic analysis. In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe, complement, or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the analyte and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze barcoded analyte. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### (j) Kits

In some instances, disclosed herein are compositions and systems that are used to carry out the methods described herein. In some embodiments, the kit includes a padlock probe or snail probe and a ligase (e.g., a T4 DNA ligase (Rnl2), a SplintR ligase, a single stranded DNA ligase, or a T4 DNA ligase). In some embodiments, the kit further includes one or more primers (e.g., two or more primers, three or more primers, four or more primers, five or more primers, six or more primers, seven or more primers, eight or more primers, nine or more primers, or ten or more primers) and a polymerase (e.g., a Phi29 DNA polymerase or other strand displacing polymerase).

In some embodiments, the kit further includes a substrate including a plurality of capture probes, where a capture probe of the plurality capture probes includes a capture domain, wherein the analyte is capable of hybridizing to the capture domain. In some embodiments, the kit includes a substrate including a plurality of capture probes, where a capture probe of the plurality of capture probes includes a capture domain and a spatial barcode. In some instances, the capture probe is specific to a target analyte. In some instances, the target analyte is Scgb1a1. In some instances, the target analyte is HPCA. It is appreciated that the kit can include any of the elements of the substrate, array, or capture probes as described herein.

In some embodiments, the kit further includes a plurality of detection probes, wherein a detection probe from the plurality of detection probes comprises a sequence that is substantially complementary to a sequence of the padlock probe or snail probe and a detectable label (e.g., any of the exemplary detectable labels described herein (e.g., a fluorophore)).

In some embodiments, a kit used to carry out the methods described herein includes:
(a) one or more padlock probes or snail probes and a ligase; (b) one or more primers and a polymerase;
(c) a substrate including a plurality of capture probes, wherein a capture probe of the plurality of capture probes includes a capture domain; and (d) instructions for performing the methods described herein.

In some embodiments, a kit used to carry out the methods described herein includes: (a) one or more padlock probes or snail probes and a ligase; (b) one or more primers and a polymerase; (c) a substrate including a plurality of capture probes, wherein a capture probe of the plurality of capture probes includes a capture domain; (d) one or more fluorescent dyes and/or one or more detection probes; and (e) instructions for performing the methods described herein.

In some embodiments, a kit used to carry out the methods described herein includes: (a) one or more padlock probes or snail probes and a ligase; (b) one or more primers and a polymerase; (c) a substrate including a plurality of capture probes, wherein a capture probe of the plurality of capture probes includes a capture domain; (d) one or more fluorescent dyes and/or one or more detection probes; (e) a uracil-DNA glycosylase enzyme; and (f) instructions for performing the methods described herein.

### EXAMPLES

### Example 1—Method for Determining the Mislocalization of an Analyte Using RCA

This example provides a method of identifying the mislocalization of an analyte (e.g., an mRNA molecule) in a biological sample (e.g., a fresh frozen tissue section) where the mRNA molecule is hybridized to a capture domain of a capture probe and the capture probe is extended using the mRNA as a template for extension. As an overview, prior to mRNA capture, the sample is stained using H&E, immunofluorescence, or immunohistochemistry. Then, the sample is imaged to provide a first image (also referred to as a “ground truth” or “baseline reference” image). After imaging, analytes (e.g., mRNA transcripts) are captured on an array comprising capture probes. The capture probe is extended using a polymerase (e.g., a reverse transcriptase) and one or more fluorescently labelled dNTPs maybe optionally incorporated into the extension product. After generating the extended capture probe, KOH is added to remove the mRNA molecule, and then either a padlock probe or a snail probe is hybridized to the extended capture probe.

The hybridized padlock probe or snail probe is ligated (e.g., using T4 DNA ligase), and the padlock probe or snail probe is amplified using rolling circle amplification (RCA) (e.g., using phi29 DNA polymerase) and a signal (e.g., fluorescence) corresponding to the amplified RCA product (e.g., microscopy puncta) is detected using labeled detection probes. A second image of the biological sample is taken to quantify and/or quantitate the signal from the detection probes hybridized to the RCA product. Comparing the first (ground truth) image to the second image after RCA product detection, one can determine the distance of mislocalization of the analyte during analyte capture on the array.

The assay further allows determination of analyte mislocalization rates as various experimental parameters are altered. For instance, permeabilization time (e.g., 0-30 minutes), permeabilization enzymes and/or concentrations, permeabilization temperature (e.g., room temperature; 37° C.), and tissue section thickness can be altered, and the effect of each experimental parameter on analyte mislocalization can be calculated.

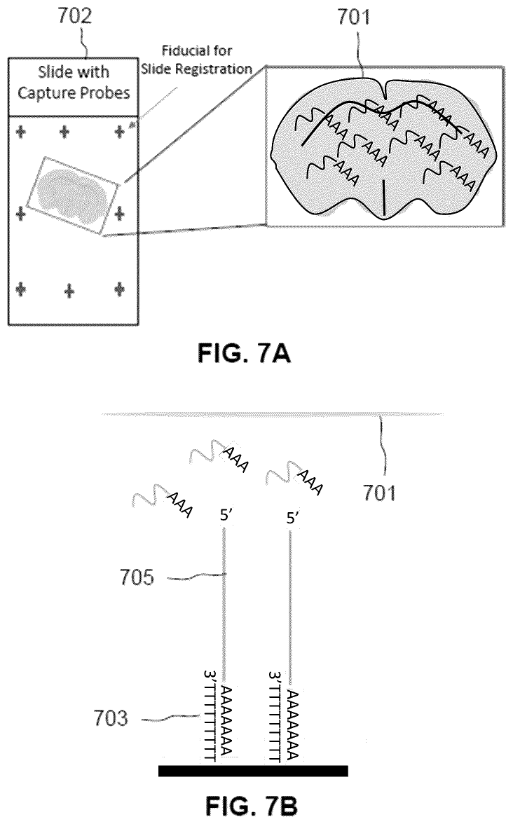

As shown in FIGS. 7A-7B, a biological sample 701 is contacted with a substrate 702 including a plurality of capture probes, where each capture probe includes a capture domain (in this example, a poly-dT sequence 703). In some instances, the probe is specific for a target such as Scgb1a1 or HPCA or any other desired target. The biological sample is permeabilized and the mRNA molecules are released from the biological sample. In particular, after 30 minutes the tissue is washed and permeabilized by adding various concentrations of proteinase K (e.g., 1 mg/ml; 1.25 mg/ml; 1.5 mg/ml Proteinase K), incubated at various temperatures (e.g., room temperature; 37° C.) for various times (e.g., 1 minute; 5minutes; 10 minutes; 20 minutes; 30 minutes). After, the samples are washed to remove the protease. The released mRNA molecules 705 are allowed to hybridize to the capture domain on the capture probe 703 immobilized on the array via the polyadenylated tail on the 3′ end of the mRNA molecule. Excess mRNA molecules not hybridized to a capture probe are washed off.

The mRNA hybridized to the capture probe is contacted with a padlock probe or a snail probe under conditions that allow the padlock probe or snail probe to hybridize to the extended capture probe.

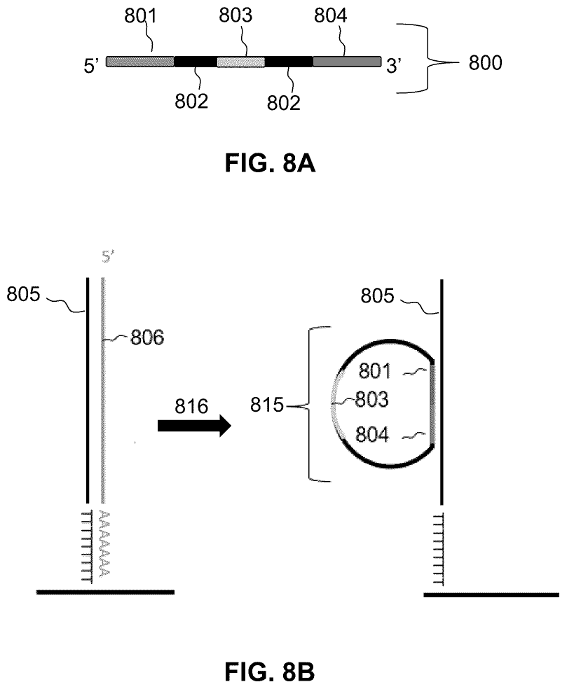

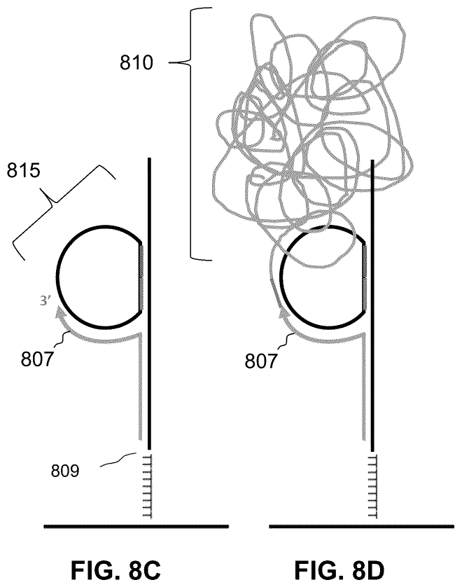

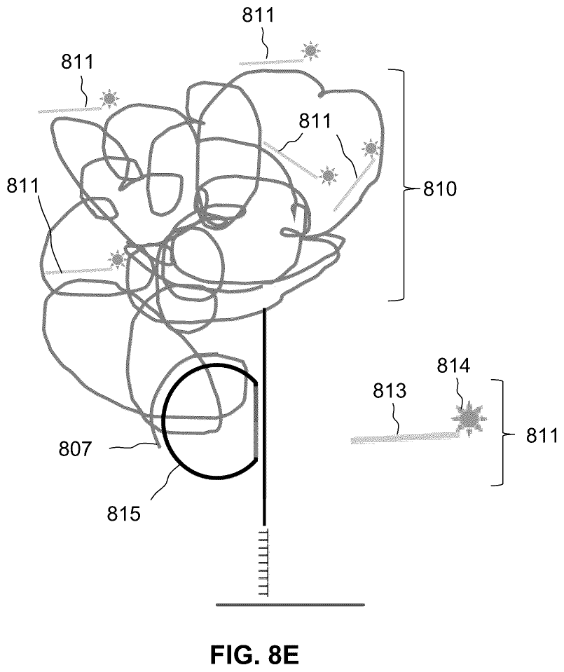

As a brief overview, a padlock probe (800 in FIG. 8A) or snail probe is hybridized to an extended capture probe molecule 815 in FIG. 8B. In some instances, a snail probe is tethered to the capture probe in order to retain the spatial location of any subsequent amplification product. The padlock probe or snail probe is ligated to create a circularized padlock probe or a circularized snail probe and amplified using RCA 810 in FIG. 8D. The amplified circularized padlock probe or snail probe is contacted with a plurality of detection probes 811 in FIG. 8E. The sample (e.g., a tissue section) is imaged (e.g., brightfield imaging to detect H&E stain; fluorescent stain for a protein marker). For example, an analyte can be associated with a stain (e.g., H&E; immunofluorescence), allowing a user to identify where the analyte is found in the biological sample.

More particularly, in some instances, the sample is decrosslinked to remove formaldehyde crosslinks within the sample thereby releasing mRNA molecules from the sample. Briefly, the tissue samples are incubated with an HCl solution for 1 minute, repeated twice for a total of 3 minutes. Following HCl incubations, the tissue sections are incubated at 70° C. for 1 hour in TE pH 9.0. TE is removed and the tissues are incubated in 1×PBS-Tween for 15 minutes.

As shown in FIG. 8A, a padlock probe 800 (or a snail probe) includes a first sequence 801 that is substantially complementary to a first portion of the extended capture probe 805, a backbone sequence 802 that includes a barcode sequence 803, and a second sequence 804 that is substantially complementary to a second portion of the extended capture probe. After capture of an analyte (e.g., mRNA molecule) 705 in FIG. 7B, the capture probe 703 (as shown in FIG. 7B) is extended using reverse transcriptase and the analyte as a template, thereby generating an extended capture probe 805. The padlock probe 800 or snail probe is added to the substrate and hybridizes to the extended capture probe 805. In this example, the first sequence and second sequence are adjacent when hybridized to the extended capture probe 805. As a result, as shown in FIG. 8B, the second sequence 804 can be directly ligated to the first sequence 801, thereby creating a circularized padlock probe 815 or snail probe.

As shown in FIG. 8C, following circularization of the padlock probe or snail probe, a primer 807 is hybridized to the circularized padlock probe 815 or snail probe. The primer 807 includes a sequence that is substantially complementary to the circularized padlock probe or snail probe and, in some instances, a sequence that is substantially complementary to the extended capture probe. In some embodiments, the primer 807 is ligated 809 to the free 3′ end of the capture probe. Rolling circle amplification (RCA), as shown in FIG. 8D, is used to amplify the circularized padlock probe 815 or snail probe using a Phi29 DNA polymerase. In some instances, the amplified circularized padlock probe 810 or snail probe remains tethered to the extended capture probe where the continuous single-stranded copies produced by RCA remains associated with the location on the substrate via the tether to the extended capture probe. In other embodiments, no snail probe is used, and amplification proceeds using the circularized padlock probe 810 as shown in FIG. 8E.

After amplification, labeled detection probes are used as a proxy to detect the analyte 811 in FIG. 8E. As shown in FIG. 8E, the continuous single-stranded copies (e.g., amplified circularized padlock probe or snail probe) of the circularized padlock probe 810 or snail probe produced by RCA are contacted with a plurality of detection probes 811, wherein a detection probe 811 includes a sequence 813 that is substantially complementary to a sequence of the padlock probe or snail probe and a fluorophore 814. Fluorescent detection of the fluorophore can be measured and the distance of the fluorescent label as compared to the analyte's original position in the biological sample can be mapped to the first image taken prior to analyte capture (i.e., the ground truth H&E or IF image).

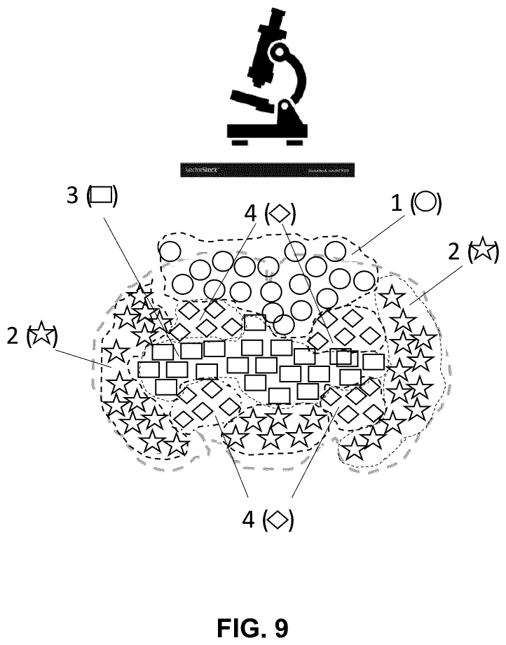

FIG. 9 shows the detected signals from a plurality of amplified circularized padlock probes or snail probe on the substrate, each numerical area (1 through 4) representing a different analyte or analyte derived molecule (each area is indicated by a dotted line and a corresponding numeral and shape). The optimal parameters for a specific tissue are determined based on identifying the experimental conditions giving rise to lowest or minimalist amount of analyte mislocalization (i.e., where the detection probe signal (e.g., from the second image) and the first image of the analyte (e.g., H&E stain) substantially overlap.

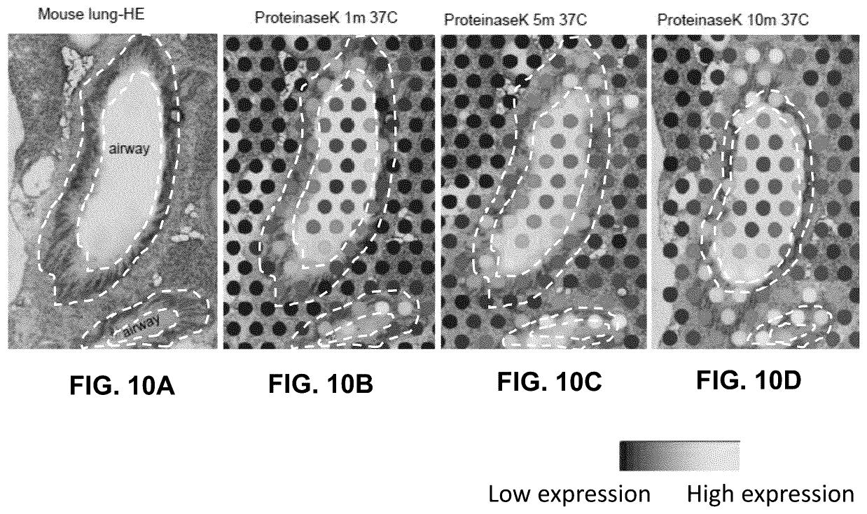

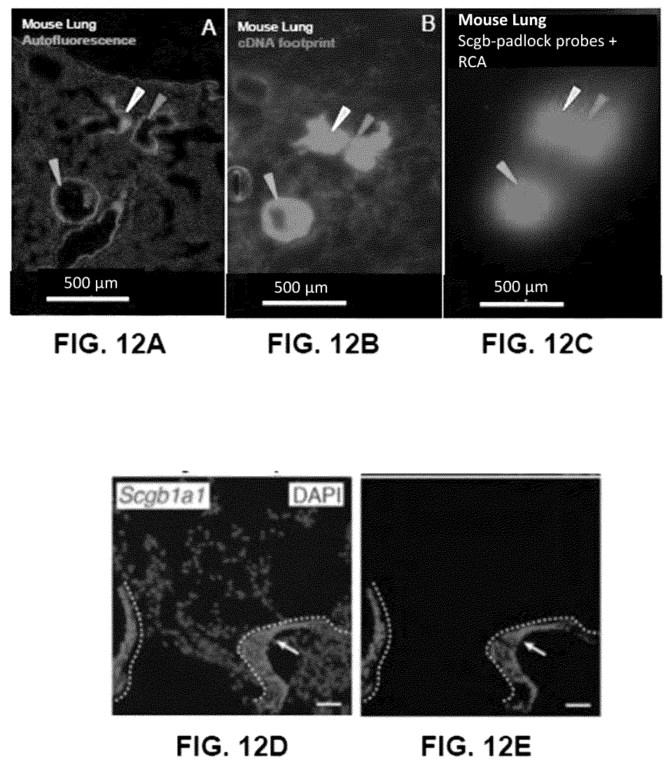

An example of mRNA transcript mislocalization is shown in FIGS. 10A-10D, which shows a gene expression heat map of Scgb1a1 mRNA molecules following different permeabilization times. Scgb1a1 is expressed only in the cells lining the airway of mouse lung (see FIGS. 12D and 12E). Here, a mouse lung tissue section was permeabilized using proteinase K at 37° C. for 1 minute, 5 minute, or 10 minutes. Referring to FIGS. 10B-10D, the gene expression heat maps showed that Scgb1a1 transcripts mislocalized from their predicted locations (i.e., diffused away from the cells lining the lung airway) within the biological sample and that the amount of mislocalization (e.g., distance travelled from its predicted location) correlated with permeabilization time, where increased permeabilization time resulted in increased analyte mislocalization. For example, in FIG. 10D there are multiple gene expression data points (green dots) having elevated gene expression at positions distal to cells lining the lung airway. In comparison, similar levels of gene expression (green dots) were found only in area in or adjacent to cells lining the lung airway in FIG. 10B. The area between the two white dashed lines in FIGS. 10A-10D indicate the predicted location for Scgb1a1 mRNA expression (i.e., the lining of the lung airway) in each of the image panes in FIGS. 10A-10D). Each image pane in FIGS. 10A-10D includes two sets of two white dashed lines, each indicating the lining of a lung airway.

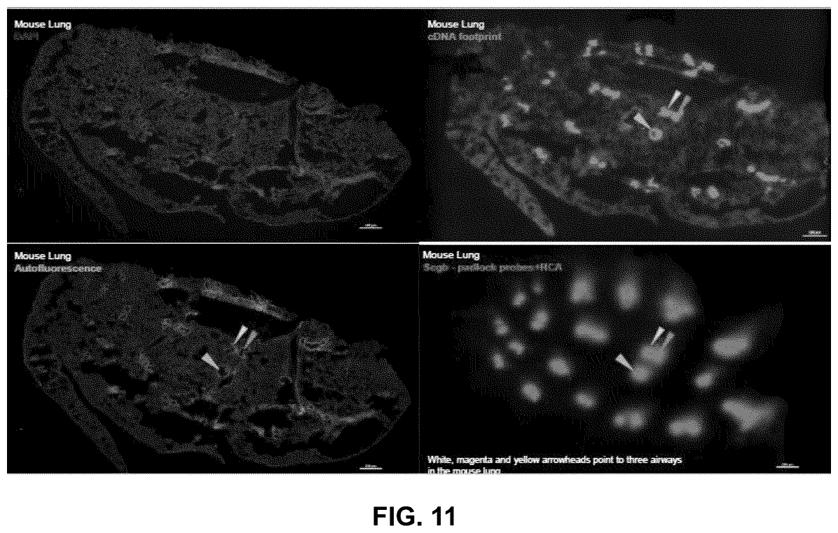

As shown in FIG. 11 and FIGS. 12A-C, the methods provided herein can be used to measure mRNA transcript mislocalization. Padlock probes were designed to hybridize to extended capture probes following capture of Scgb1a1 mRNA transcripts on the array. Following padlock probe hybridization and circularization, RCA was used to amplify the circularized padlock probe. Fluorescent probes having a sequence at least substantially complementary to a portion of the amplified circularized padlock probe were added to the sample to detect the RCA product. The RCA product serves as a proxy for the location of Scgb1a1 mRNA molecules and thus any mislocalization of the Scgb1a1 mRNA molecules from their original position in the biological sample.

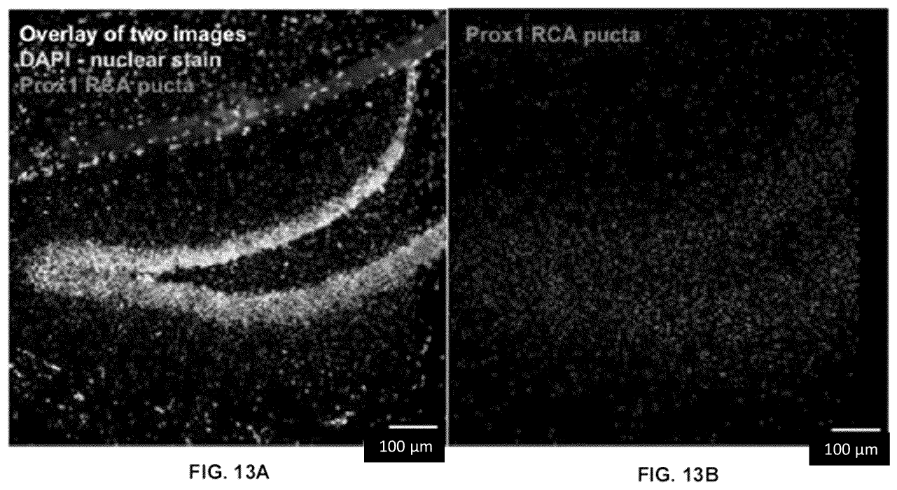

FIG. 11 shows the signal from the fluorescent probes hybridized to the RCA product (bottom right panel) as compared to nuclear staining of the biological sample (DAPI, upper left panel), auto fluorescence of the biological sample (bottom left panel), and a fluorescent cDNA footprint (e.g., incorporation of one or more fluorescent nucleotides into extended capture probes that hybridize to mRNAs) (upper right panel). The arrows point to three different airways in the mouse lung. FIGS. 12A-12C show magnified images of the three lung airways highlighted in FIG. 11. FIG. 12D and FIG. 12E show single molecule Fluorescent In Situ Hybridization (smFISH) images for Scgb1a1 mRNA molecules confirming that the Scgb1a1 mRNA molecules were detected only in the cells lining the airway. The data from FIGS. 12A-12C was then used to calculate the mislocalization of the Scgb1a1 mRNA molecules following different permeabilization conditions. In a second experiment, mislocalization of Prox1, which encodes for Prospero homeobox protein 1 (PROX1), was examined in fresh frozen mouse brain sections. 10 μm sections were placed on tissue optimization slides (Visium Tissue Optimization Slides, 10× Genomics, Pleasanton, CA). The fresh frozen mouse brain samples were fixed in methanol for 20 minutes at −20° C. The mouse brain samples were stained with DAPI for nuclear detection (or alternately with H&E) and imaged. The sample was treated with a permeabilization buffer comprising pepsin or proteinase K to allow migration of analytes from the mouse brain sample to the tissue optimization slides. mRNA was captured on the capture probes of the tissue optimization slide, and a reverse transcription reaction was performed to convert the captured mRNA into cDNA. After, the tissue optimization slide was treated with 0.8M KOH to denature and remove the mRNA from the capture probes. Snail probes for Prox1 cDNA were hybridized to the generated cDNA molecules affixed to the tissue optimization slide, followed by ligation, rolling circle amplification, and detection of the RCA product using fluorescently-tagged probes hybridized to the RCA product. The results are shown in FIG. 13A (overlay of DAPI and Prox1 using RCA) and FIG. 13B (Prox1 using RCA only). Using RCA, detection of Prox1 was seen outside areas of DAPI stain, demonstrating in this example, the Prox1 mRNA transcripts had mislocalized during mRNA capture on the tissue optimization slide. Using location and intensity of the RCA product signal, one can determine (e.g., calculate) an average mislocalization distance of Prox1 after RCA.

The data in the above experiments show that methods described herein can be used to measure mislocalization of analytes, including mRNA transcripts. In particular, these data show that the methods described herein can be used to determine analyte mislocalization as various experimental parameters are altered (e.g., permeabilization time (e.g., 0-30 minutes), permeabilization concentration, permeabilization temperature (e.g., room temperature; 37° C.).

### Example 2—Method for Determining the Mislocalization of an Analyte Using Templated Ligation

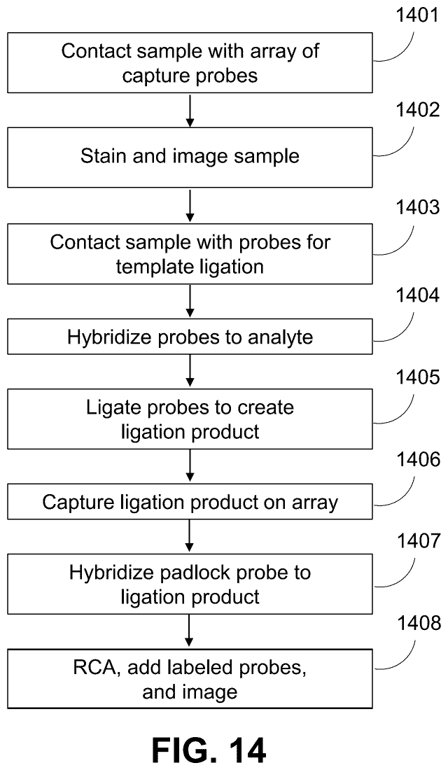

This example provides a method of identifying the mislocalization of an analyte (e.g., an mRNA molecule) in a biological sample (e.g., a tissue section) using a templated ligation product as a proxy for detection of the analyte. Here, a padlock probe or snail probe hybridizes directly to the ligation product after the ligation product hybridizes to a capture domain of a capture probe on an array (e.g., tissue optimization slide). Briefly, as shown in FIG. 14, the methods include 1401 contacting a biological sample with array of capture probes. The sample is stained (e.g., using H&E, immunofluorescence, or immunohistochemistry), and imaged 1402. Samples are destained (e.g., using HCl) and decrosslinked. Following decrosslinking, samples are treated with pre-hybridization buffer (e.g., hybridization buffer without the first and second probes). Then, the biological sample 1403 is contacted with a first probe and a second probe, wherein the first probe and the second probe each include one or more sequences that are substantially complementary to sequences of the analyte, and wherein the second probe includes a capture probe capture domain. The first probe and the second probe 1404 hybridize to complementary sequences in the analyte. After hybridization, a ligation product comprising the first probe and the second probe 1405 is generated, and the ligation product is released from the biological sample (e.g., using RNAse H). The biological sample is permeabilized and the liberated ligation product is then available 1406 to hybridize to the capture domain of a probe on the array (e.g., Visium Tissue Optimization or Visium Gene Expression slide). A padlock probe (e.g., 800 in FIG. 8A) or snail probe hybridize 1407 directly to the captured ligation product. Optionally, a snail probe is tethered to the capture probe. The padlock probe or snail probe is ligated to create a circularized padlock probe or snail probe and amplified using RCA.

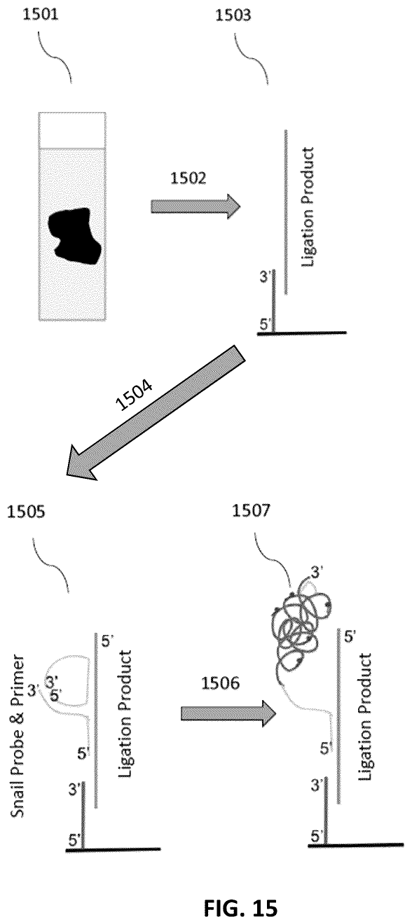

The method described here is depicted in FIG. 15. In 1501, a biological sample (e.g., a tissue section) is placed on a slide comprising capture probes (e.g., Visium Tissue

Optimization or Visium Gene Expression slide), stained and imaged. After imaging, template ligation probes are added to the biological sample, where the probes hybridize to a target and are ligated 1502. After permeabilization of the biological sample, the ligation product is captured 1503 on the slide (e.g., hybridized to the capture domain of a capture probe). A snail probe and primer is added 1504 to the array, where the snail probe hybridizes 1505 to the captured ligation product. After padlock probe or snail probe ligation, rolling circle amplification 1506 is performed, and labeled probes are added 1507 to hybridize to the RCA product.

Then, the sample is imaged for a second time 1408. One can measure the distance of an analyte imaged in 1402 to the distance of the analyte imaged in 1408 (e.g., via overlay of the images) to determine mislocalization of the analyte.

### Example 3—Method for Determining the Mislocalization of a Protein Analyte

This example provides a method of identifying the mislocalization of an analyte (e.g., a protein molecule) in a biological sample (e.g., a tissue section) after (1) binding of an analyte capture agent to a protein and (2) capture of an oligonucleotide of the analyte capture agent by a capture probe on an array.

Briefly, a formalin-fixed, paraffin-embedded tissue sample is sectioned and mounted on an array slide (e.g., Visium Gene Expression Slides, 10× Genomics, CA) comprising capture probes, and is deparaffinized using a series of xylene and ethanol washes prior to drying at room temperature. Next, the array slides are heated, followed by a series of ethanol washes (e.g., 100%, 96%, 96%, and 70% ethanol). The tissue section is H&E stained and brightfield imaged. Alternatively, tissues can be stained (e.g., immunofluorescence stained) instead of H&E staining.

After staining, the tissue section is washed and decrosslinked. After decrosslinking, analyte capture agents are added to the tissue section to allow the analyte capture agents to bind to protein targets in the tissue section. After washing to remove unbound analyte capture agents, the tissue section is permeabilized and the oligonucleotide of the analyte capture agent hybridizes to a capture domain of the capture probes on the array. Optionally, the analyte binding agent oligonucleotides are extended to create an extended capture probe.

Then, a padlock probe (e.g., 800 in FIG. 8A) or snail probe is hybridized either directly to oligonucleotide of the analyte capture agent or to the extended capture probe. A second snail probe is tethered to the extended capture probe. The padlock probe or snail probe is ligated to create a circularized padlock probe or snail probe and amplified using RCA. Then, the amplified circularized padlock probe or snail probe is contacted with a plurality of detection probes. The sample (e.g., a tissue section) is imaged for a second time (e.g., brightfield imaging to detect H&E stain; fluorescent stain for a protein marker). For example, an analyte can be associated with a stain (e.g., H&E; immunofluorescence), allowing a user to identify where the analyte is located in the biological sample. The second image can be compared to the first image generated prior to addition of the analyte capture agents. By comparing the two images, one can measure mislocalization of the protein analyte using the analyte capture agent.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method comprising:
(a) obtaining a first image of a tissue sample;
(b) hybridizing a first analyte to a capture domain of a capture probe on an array, wherein the array comprises a plurality of capture probes, wherein the plurality of capture probes comprise a capture domain;
(c) extending the capture probe using the first analyte as a template, thereby generating an extended capture probe on the array;
(d) hybridizing a padlock probe or a snail probe to the extended capture probe on the array;
(e) circularizing the padlock probe or the snail probe on the array;
(f) amplifying the padlock probe or the snail probe on the array, thereby generating an amplified circularized padlock probe or an amplified circularized snail probe on the array;
(g) hybridizing a plurality of detection probes to the amplified circularized padlock probe or the amplified circularized snail probe on the array, wherein a detection probe from the plurality of detection probes comprises:
a nucleic acid sequence that is substantially complementary to a sequence of the padlock probe or the snail probe, and
a detectable label; and
(h) obtaining a second image of the tissue sample and detecting a signal intensity from the plurality of detection probes in the second image.

2. The method of claim 1, wherein the tissue sample is stained with a biomarker that is specific for a second analyte having colocalized expression with the first analyte.

3. The method of claim 2, further comprising determining mislocalization of the first analyte within the tissue sample by comparing detection of the biomarker in the first image to the signal intensity of the detectable label in the second image.

4. The method of claim 1, wherein the padlock probe or the snail probe comprises:
(i) a first sequence that is substantially complementary to a first portion of the extended capture probe, or a complement thereof;
(ii) a backbone sequence; and
(iii) a second sequence that is substantially complementary to a second portion of the extended capture probe, or a complement thereof.

5. The method of claim 4, wherein the first sequence and the second sequence are substantially complementary to adjacent sequences of the extended capture probe, or a complement thereof.

6. The method of claim 1, wherein the detectable label comprises a fluorophore.

7. The method of claim 1, wherein the tissue sample is permeabilized using a permeabilization agent comprising proteinase K or pepsin.

8. The method of claim 1, wherein the tissue sample comprises a formalin-fixed paraffin embedded sample or a fresh frozen sample.

9. The method of claim 1, further comprising varying (i) thickness of the tissue sample, (ii) permeabilization time and/or (iii) permeabilization temperature to determine an optimal permeabilization time for minimizing nucleic acid mislocalization in the tissue sample.

10. The method of claim 3, wherein determining the mislocalization of the nucleic acid comprises measuring signal intensity from the plurality of detection probes in the second image and comparing the signal intensity to detection of the biomarker in the first image of the tissue sample.

11. A method comprising:
(a) obtaining a first image of a tissue sample;
(b) hybridizing an analyte-derived molecule to a capture domain of a capture probe on an array, wherein the array comprises a plurality of capture probes, wherein the plurality of capture probes comprise a capture domain;
(c) extending the capture probe using the analyte-derived molecule as a template, thereby generating an extended capture probe on the array;
(d) hybridizing a padlock probe or a snail probe to the extended capture probe on the array;
(e) circularizing the padlock probe or the snail probe on the array;
(f) amplifying the padlock probe or the snail probe on the array, thereby generating an amplified circularized padlock probe or an amplified circularized snail probe on the array;
(g) hybridizing a plurality of detection probes to the amplified circularized padlock probe or the amplified circularized snail probe on the array, wherein a detection probe from the plurality of detection probes comprises:
a nucleic acid sequence that is substantially complementary to a sequence of the padlock probe or the snail probe, and
a detectable label; and
(h) obtaining a second image of the tissue sample and detecting a signal intensity from the plurality of detection probes in the second image.

12. The method of claim 11, further comprising determining mislocalization of the analyte-derived molecule within the tissue sample by comparing detection of a biomarker in the first image to the signal intensity of the detectable label in the second image.

13. The method of claim 11, wherein the analyte-derived molecule is a ligation product, wherein the method further comprises, prior to step (b):
contacting a plurality of first probes and second probes to the tissue sample on the array,
wherein the plurality of first probes and second probes target a plurality of nucleic acids in the tissue sample,
wherein a first probe and a second probe of the plurality comprise sequences that are substantially complementary to the analyte, wherein the analyte is a target nucleic acid, and
wherein the second probe comprises a capture probe capture domain sequence that is complementary to all or a portion of the capture domain;
hybridizing the first probe and the second probe to the target nucleic acid;
ligating the first probe and the second probe to generate a ligation product; and
releasing the ligation product from the target nucleic acid.

14. The method of claim 13, wherein the first probe and the second probe are substantially complementary to adjacent sequences of the target nucleic acid.

15. The method of claim 13, where the first probe and the second probe hybridize to sequences that are not adjacent to each other on the target nucleic acid, and wherein the first probe is extended with a DNA polymerase, thereby (i) filling in a gap between the first probe and the second probe and (ii) generating an extended first probe.

16. The method of claim 13, wherein the first probe and/or the second probe is a DNA probe.

17. The method of claim 13, further comprising releasing the ligation product from the target nucleic acid comprises contacting the tissue sample with an endoribonuclease, optionally wherein the endoribonuclease is an RNase H enzyme.

18. The method of claim 11, wherein the analyte-derived molecule is an analyte capture agent.

19. The method of claim 18, further comprising, prior to step (b):
contacting the tissue sample with a plurality of analyte capture agents, wherein the analyte capture agent comprises an analyte binding moiety and an oligonucleotide comprising an analyte binding moiety barcode and an analyte capture sequence, wherein the analyte capture sequence comprises a sequence complementary to the capture domain of the capture probe on the array; and
binding the analyte binding moiety of the analyte capture agent to the analyte, wherein the analyte is a protein.

20. A system comprising:
(a) an array comprising capture probes, wherein a capture probe of the plurality comprises a capture domain;
(b) a plurality of padlock probes and/or snail probes, wherein a padlock probe or a snail probe of the plurality of padlock probes and/or snail probes comprises:
(i) a first sequence that is substantially complementary to a first portion of an analyte or an analyte-derived molecule, or complements thereof,
(ii) a backbone sequence, and
(iii) a second sequence that is substantially complementary to a second portion of the analyte, the analyte-derived molecule, or complements thereof;
(c) a ligase, a polymerase, a reverse transcriptase, or a combination thereof; and
(d) a plurality of detection probes.
